(19) United States
(12) Patent Application Publication
(10) Pub. No.: US 2002/0037286 A1
(43) Pub. Date: Mar. 28, 2002

(54) Methods for altering T cell and macrophage activation

(76) Inventors: Matthias Krause, Cambridge, MA (US); Antonio S. Sechi, Wendenburg (DE); Frank B. Gertler, Boston, MA (US); Jurgen Wehland, Bad Harzburg (DE)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/825,144

(22) Filed: Apr. 3, 2001

Related U.S. Application Data
(60) Provisional application No. 60/194,215, filed on Apr. 3, 2000.

(51) Int. Cl. A61K 39/395; C07K 16/30

(52) U.S. Cl. 424/155.1; 424/178.1; 530/388.3; 530/391.1

(57) ABSTRACT

The invention relates to polypeptides which form a protein complex which regulates cytoskeletal reorganization, for example, in connection with T cell activation. The invention also relates to nucleic acids that encode the polypeptides, as well as agents which bind the nucleic acids or the encoded polypeptides. Also disclosed are methods of using such nucleic acids, polypeptides and binding agents to modulate protein complex formation, actin cytoskeletal reorganization and cell activity, as well as drug discovery methods.

### RELATED APPLICATION

This application claims priority under 35 U.S.C. §119 from U.S. provisional application Ser. No. 60/194,215, filed Apr. 3, 2000.

### GOVERNMENT SUPPORT

This work was funded in part by the National Institutes of Health under grant number GM58801. The government may retain certain rights in this invention.

### FIELD OF THE INVENTION

This invention relates to polypeptides which form a protein complex which interacts with the T cell receptor and regulates cytoskeletal reorganization in connection with T cell activation. The invention also relates to nucleic acids that encode the polypeptides, as well as agents which bind the nucleic acids or the encoded polypeptides. The invention further relates to methods of using such nucleic acids, polypeptides and binding agents to modulate protein complex formation and T cell activity, as well as drug discovery methods.

### BACKGROUND OF THE INVENTION

T cells play a central role in immune responses mounted against pathogens and cancer. It has been asserted that dramatic cytoskeletal reorganization during T cell activation is a prerequisite for a specific immune response (see, e.g., Penninger and Crabtree, Cell 96(l):9-12, 1999). Upon binding to an antigen presenting cell (APC), the cytoskeleton of a T cell rapidly polarizes. The accumulation of actin in a tight collar at the T cell/APC interface has been reported to stabilize a continuous contact between T cells and APCs (Ryser et al., 1982; Valitutti et al., 1995). The formation of this tight contact is accompanied by the reorientation of the microtubule-organizing center (MTOC) towards the contact site to ensure a polarized release of cytokines or cytotoxic factors (Geiger et al., 1982; Kupfer et al., 1987; Kupfer et al., 1991). Although actin remodeling is reported to be important for T cell activation (see Penninger and Crabtree, 1999), it is not known how T cell receptor (TCR) signaling is linked to the rearrangement of the actin cytoskeleton.

Modulation of T cell activity is important in treatment of various conditions. For example, treatment of autoimmune diseases would be aided by the ability to downregulate T cell activity by therapeutic intervention. Conversely, treatment of conditions in which an enhanced immune response would be beneficial, such as infections and cancer, would be aided by increased T cell activity. Accordingly, there is a need for methods and compositions for effectively modulating T cell activity.

### SUMMARY OF THE INVENTION

It now has been discovered that Fyb/SLAP proteins are ligands for the EVH1 domains of Ena/VASP proteins. Furthermore, a second human Fyb/SLAP protein, termed Fyb/SLAP2, has been identified. In contrast to other known EVH1 ligands, Fyb/SLAP proteins are localized to lamellipodia of spreading platelets and colocalized in T cells with F-actin, Ena/VASP proteins, Vav, WASP and the Arp2/3 complex. Modulation of Fyb/SLAP binding to Ena/VASP proteins can be used in the modulation of actin cytoskeleton reorganization to regulate hematopoietic cell activity.

According to one aspect of the invention, methods for inhibiting cytoskeletal rearrangement in a cell or cell fragment are provided. The methods include contacting the cell or cell fragment with an amount of a Fyb/SLAP complex inhibitor sufficient to inhibit the formation of a complex of an Ena/VASP protein and a Fyb/SLAP protein. In certain embodiments, the Fyb/SLAP complex inhibitor binds to the EVH1 domain of the Ena/VASP protein and inhibits binding of the Ena/VASP protein to a Fyb/SLAP protein. In preferred embodiments, the Ena/VASP family protein is selected from the group consisting of Ena, Mena, VASP and Ev1. In other embodiments, the Fyb/SLAP complex inhibitor comprises the peptide FPPPP (SEQ ID NO: 15) or a peptide mimetic having an equivalent binding specificity. An “equivalent binding specificity” means that the mimetic competes for binding with the peptide FPPPP (SEQ ID NO:15). Preferably there is at least one acidic amino acid residue on one or both sides of the peptide. In other embodiments, the Fyb/SLAP complex inhibitor is a reverse charged variant in which there is at least one basic amino acid on one or both sides of the peptide (which complements an acidic charge on a variant EVH1 domain). Preferably, the Fyb/SLAP complex inhibitor is selected from the group consisting of ActA repeats, EVH1 binding peptides, ScarWA, and dominant negative Fyb/SLAP fragments.

In still other embodiments, the Fyb/SLAP complex inhibitor binds a Fyb/SLAP protein and inhibits binding of an Ena/VASP protein to the Fyb/SLAP protein. In certain preferred embodiments, the Fyb/SLAP complex inhibitor is a polypeptide which has a functional EVH1 domain but which cannot function as an Ena/VASP protein in cytoskeletal rearrangement of the cell or cell fragment. These include nonfunctional Ena/VASP proteins such as Ena/VASP fragments and peptides containing an EVH1 domain. In other preferred embodiments, the Fyb/SLAP complex inhibitor is an antibody or antibody fragment which binds Fyb/SLAP.

In other embodiments, the Fyb/SLAP complex inhibitor is an antisense nucleic acid molecule which inhibits the expression of a Fyb/SLAP protein or an Ena/VASP protein.

In certain embodiments the Fyb/SLAP complex inhibitor is administered in vivo to a subject in need of such treatment.

In other embodiments the cell is a lymphocyte, preferably a T cell, or a macrophage. In still other embodiments, the cell fragment is a platelet.

According to another aspect of the invention, methods for inhibiting cytoskeletal rearrangement in a cell or cell fragment are provided. The methods include inhibiting the expression of a Fyb/SLAP protein sufficiently to inhibit the formation of a complex of an Ena/VASP protein and a Fyb/SLAP protein.

According to still another aspect of the invention, methods for enhancing cytoskeletal rearrangement in a cell or cell fragment are provided. The methods include contacting the cell or cell fragment with an amount of a composition which increases the amount of a Fyb/SLAP polypeptide in the cell or cell fragment sufficient to enhance the formation of a complex of an Ena/VASP protein and a Fyb/SLAP protein. In certain embodiments the complex is formed upon stimulation of the cell or cell fragment. In preferred embodiments, the composition is a Fyb/SLAP polypeptide, a fusion polypeptide comprising Fyb/SLAP amino acids or a nucleic acid molecule which encodes a Fyb/SLAP polypeptide. In other embodiments, the composition which increases the amount of a Fyb/SLAP polypeptide is administered to a subject in need of such treatment in vivo.

In some embodiments, the cell is a lymphocyte, preferably a T cell, or a macrophage. In other embodiments the cell fragment is a platelet.

According to yet another aspect of the invention, methods for increasing a T cell response to T cell receptor stimulation are provided. The methods include contacting a T cell with a Fyb/SLAP complex activator sufficient to promote the formation of a complex of an Ena/VASP protein and a Fyb/SLAP protein in the T cell. In some embodiments, Fyb/SLAP complex activators include Fyb/SLAP proteins, fusion proteins containing appropriate Fyb/SLAP domains and nucleic acid molecule which encode a Fyb/SLAP polypeptide.

In certain embodiments, the Fyb/SLAP activator is administered in vivo to a subject in need of such treatment. In preferred embodiments, the subject has or is at risk of developing an infectious disease or cancer.

According to another aspect of the invention, methods for inhibiting a T cell response to T cell receptor stimulation are provided. The methods include contacting a T cell with an amount of one of the foregoing Fyb/SLAP complex inhibitors sufficient to inhibit formation of a complex of a Fyb/SLAP protein and an Ena/VASP protein in the T cell.

In certain embodiments, the Fyb/SLAP inhibitor is administered in vivo to a subject in need of such treatment. In preferred embodiments, the subject has or is at risk of developing an autoimmune disease or a condition characterized by inflammation.

According to yet another aspect of the invention, methods for increasing platelet aggregation are provided. The methods include contacting a platelet with one of the foregoing Fyb/SLAP complex inhibitors to inhibit formation of a complex of a Fyb/SLAP protein and an Ena/VASP protein in the platelet. In certain embodiments, the Fyb/SLAP inhibitor is administered in vivo to a subject in need of such treatment. In other embodiments, the administration of the Fyb/SLAP inhibitor increases wound healing or clotting.

Compositions which include an effective amount of one of the foregoing Fyb/SLAP complex inhibitors and a pharmaceutically acceptable carrier are provided according to another aspect of the invention. Compositions comprising an effective amount of one of the foregoing Fyb/SLAP complex activators and a pharmaceutically acceptable carrier also are provided.

Also provided according to other aspects of the invention are isolated antibodies which selectively bind human Fyb/SLAP 2 but not Fyb/SLAP 1, and preferably the antibodies binds to SEQ ID NO:7 or to an epitope defined by amino acids 637-682 of SEQ ID NO:4. Other antibodies provided in accordance with an aspect of the invention antibodies which selectively binds Arp3 polypeptide; preferably the antibody binds to an epitope formed by the amino acids set forth in SEQ ID NO: 11.

According to another aspect of the invention, isolated human Fyb/SLAP2 polypeptides comprising the amino acids 637-682 of SEQ ID NO:4 are provided. In certain embodiments, the isolated polypeptides include fragments of the amino acid sequence of SEQ ID NO:4 which include amino acids 637-682. In one preferred embodiment the polypeptide includes the amino acid sequence of SEQ ID NO:4. Also provided are isolated nucleic acid molecules which encode the forgoing human Fyb/SLAP2 polypeptides. In certain preferred embodiments, the nucleic acid molecule comprises SEQ ID NO:5, particularly nucleotides 1939-2076. Also provided by the invention are expression vectors comprising the foregoing isolated nucleic acid molecules, operably linked to a promoter, as well as host cells transformed or transfected with the expression vectors.

According to still another aspect of the invention, methods for identifying lead compounds for a pharmacological agent useful in the treatment of disease associated with Fyb/SLAP-Ena/VASP complex formation are provided. The methods include forming a mixture comprising a Fyb/SLAP protein or EVH1 domain binding fragment thereof, a Ena/VASP protein or Fyb/SLAP binding fragment thereof, and a candidate pharmacological agent. The mixture is incubated under conditions which, in the absence of the candidate pharmacological agent, permit a first amount of specific binding of the Fyb/SLAP protein or EVH1 domain binding fragment thereof and the Ena/VASP protein or Fyb/SLAP binding fragment thereof. The methods also include detecting a test amount of the specific binding of the Fyb/SLAP protein or EVH1 domain binding fragment thereof and the Ena/VASP protein or Fyb/SLAP binding fragment thereof. An increase in the test amount of specific binding in the presence of the candidate pharmacological agent relative to the first amount of specific binding indicates that the candidate pharmacological agent is a lead compound for a pharmacological agent which increases specific binding of the Fyb/SLAP protein or EVH1 domain binding fragment thereof and the Ena/VASP protein or Fyb/SLAP binding fragment thereof. A decrease in the test amount of specific binding in the presence of the candidate pharmacological agent relative to the first amount of specific binding indicates that the candidate pharmacological agent is a lead compound for a pharmacological agent which decreases specific binding of the Fyb/SLAP protein or EVH1 domain binding fragment thereof and the Ena/VASP protein or Fyb/SLAP binding fragment thereof.

According to yet another aspect of the invention, additional methods for identifying lead compounds for a pharmacological agent useful in the treatment of disease associated with Fyb/SLAP-Ena/VASP complex formation are provided. The methods include contacting a Fyb/SLAP protein or EVH1 domain binding fragment thereof with a candidate pharmacological agent, and determining the binding of the candidate pharmacological agent to the Fyb/SLAP protein or EVH1 domain binding fragment thereof.

Kits which include the compositions also are provided, optionally including instructions for administering the compositions to modulate an immune response. The kits can also include other components, such as other medicaments, and the like.

Still other aspects of the invention provide for the use of the foregoing compositions in the preparation of a medicament. Preferred medicaments include those which are useful in modulation of an immune response in a subject.

These and other objects of the invention will be described in further detail in connection with the detailed description of the invention.

### BRIEF DESCRIPTION OF THE DRAWINGS

FIG. 1.

FIG. 1A shows a schematic representation of the full-length Fyb/SLAP2, the EST clone (IMAGE clone ID 221953; RZPD IMAGp998F02441), the murine clone 5/7, and the three GST-fusion proteins encoding for different regions of Fyb/SLAP2 (Fyb/SLAP I, III, and IX). Potential EVH1 domain-binding sites, SH3-like domain, and the 138-bp insertion are also shown.

FIG. 1B indicates that RT-PCR of the carboxy-terminus of Fyb/SLAP as obtained from HL60 cells shows two bands running at about 700 and 840 bp (lanes 1-3, RT-PCRs of three independent experiments; lane 4, 100-bp marker).

FIG. 1C depicts the results of immunodetection of Fyb/SLAP in extracts of human platelets and Jurkat T-cells. The affinity purified polyclonal antibody #51 specifically reacts with a single band on Jurkat T-cells lysate and with two bands on platelet extract whereas the polyclonal antibody #81 reacts with a single band on platelet extract.

FIG. 1D shows the single letter amino acid sequence and structural motifs of Fyb/SLAP2. Boxes represent potential EVH1 domain-binding sites, stretch of bold-faced amino acids represents the insertion not present in Fyb/SLAP1. Potential SH3-binding regions (thin underlines), potential tyrosine phosphorylation sites (bold and boxed), and the SH3-like domain (dotted underline) are shown. The sequence of Fyb/SLAP2 is available from GenBank database under the accession number AF198052.

FIG. 2.

FIG. 2A depicts the alignment of protein sequences of the amino-terminus of ActA (from L. monocytogenes), iActA (from L. ivanovii), and Fyb/SLAP. Identical amino acids are indicated with bold letters; homologous amino acids are underlined. Amino acids are numbered according to Domann et al. (1992) for ActA and to Kreft et al. (1995) for iActA.

FIG. 2B shows the alignment of the G-actin-binding sites of thymosin β4, villin, dematin, the potential G-actin binding site of Mena and Fyb/SLAP. Amino acids involved in the binding to G-actin are indicated with bold letters; homologous amino acids are underlined.

FIG. 3.

FIG. 3 shows the results of a blot overlay assay of in vitro translated [₃₅S]-labeled VASP on GST-fusion proteins of Fyb/SLAP and the murine clone 5/7. Lane 1: GST-ActA proline-rich repeats (positive control); lane 2: Fyb/SLAP I GST; lane 3: Fyb/SLAP III GST; lane 4: Fyb/SLAP IX GST; lane 5: murine clone 5/7 GST. Note that bands representing degradation products are visible in lanes 1, 4, and 5.

FIG. 3B depicts peptide spot analysis of the full-length Fyb/SLAP I and of the carboxy-terminus of Fyb/SLAP2. Peptides were synthesized on a cellulose membrane and incubated with [³⁵S]-labeled VASP. Positive spots were detected with a phosphorimager.

FIG. 3C shows the single letter amino acids sequences of the peptides corresponding to the positive spots shown in FIG. 3B.

FIG. 3D depicts the results of co-immunoprecipitation of Fyb/SLAP and VASP from a lysate of Jurkat T-cells. Extracts of activated Jurkat T-cells were incubated with monoclonal antibodies against VASP. Immune complexes were collected using anti mouse IgG agarose. Bound proteins were separated by SDS-PAGE, blotted and probed with the affinity purified antibody #51 to Fyb/SLAP. Lane 1: lysate of activated Jurkat T-cells (positive control); lane 2: proteins immunoprecipitated using the monoclonal antibody (358C1) raised against a bacterial protein; lane 3: proteins immunoprecipitated using anti-VASP specific monoclonal antibodies. Molecular weight markers are indicated on the left in FIG. 3A and FIG. 3D.

### DETAILED DESCRIPTION OF THE INVENTION

Fyb/SLAP proteins have now been identified as EVH1 domain binding proteins in hematopoietic cells. Thus Fyb/SLAP proteins can act to localize Ena/VASP proteins to the cell membrane of hematopoietic cells, thereby influencing the regulation of actin dynamics and remodeling of the actin cytoskeleton in hematopoietic cells. Fyb/SLAP2 has been identified as a new isoform of the Fyb/SLAP 1 protein (also known as Fyb-120 or SLAP-130), having an insert of 46 amino acids in the C-terminal portion of Fyb/SLAP 1.

Also identified are protein complexes which participate in and regulate cytoskeletal remodeling by altering actin dynamics. Previous investigations using the Listeria model system have demonstrated that at least two host cell components, Ena/VASP proteins and Arp2/3 complex, are required for an efficient actin tail formation. The invention also relates to the demonstration herein that both of these components participate in efficient actin rearrangement in activated T cells. Fyb/SLAP proteins and certain other proteins, such as SLP-76, Nck and WASP, form a complex to bring together Ena/VASP proteins and Arp2/3 complex. This macromolecular assembly links T cell receptor signaling to the remodeling of the actin cytoskeleton.

The invention involves in one aspect methods for modulating cytoskeletal rearrangement in cells (including cell fragments) by decreasing or increasing the interaction of the proteins in the foregoing protein complex. By altering the formation or dissolution of the complex one can modulate the remodeling of the actin cytoskeleton to modulate cytoskeletal rearrangement in cells or cell fragments. This can be useful in the modulation of hematopoietic cell activity, e.g., in regulating T cell activation.

As used herein, “modulate,” “modulated” or “modulating” refers to regulation of a property or activity of molecules or cells in a negative or positive manner. For example, positive regulation of complex formation means to increase or enhance complex formation. Conversely, negative regulation of complex formation means to inhibit or decrease complex formation.

The aforementioned Fyb/SLAP-containing protein complexes can be inhibited, for example, by contacting the cell or cell fragment with an amount of a Fyb/SLAP complex inhibitor sufficient to inhibit the formation of a complex of an Ena/VASP protein and a Fyb/SLAP protein. “Ena/VASP proteins” include proteins having an EVH1 domain, including Ena, Mena, VASP, Ev1 and fragments thereof which contain at least one EVH1 domain (see, e.g., Carl et al., Curr. Biol.9:715-718, 1999). “Fyb/SLAP proteins” refer to Fyb/SLAP1 (FYB-120; SLAP-130), Fyb/SLAP2 (FYB-130), and fragments thereof which bind to EVH1 domains.

Thus Fyb/SLAP complex inhibitors include molecules which bind to the EVH1 domain of an Ena/VASP protein and inhibits binding of the Ena/VASP protein to a Fyb/SLAP protein. Such molecules include antibodies, antibody fragments and other polypeptide binding molecules. Preferred molecules include the peptide FPPPP (SEQ ID NO: 15) or a peptide mimetic having an equivalent binding specificity. More preferred are polypeptides having the FPPPP (SEQ ID NO: 15) motif flanked by at least one acidic amino acid residue. Such larger motifs are known to occur naturally in proteins including ActA (ActA repeats) and Fyb/SLAP. The acidic residue on one or both sides of the FPPPP (SEQ ID NO: 15) motif is known to enhance binding to EVH1 domains. The invention also includes the use of reverse charged variants of the FPPPP/EVH1 domain binding pairs which naturally exist or be engineered to provide different binding specificities.

Still other Fyb/SLAP complex inhibitors are molecules which bind to a Fyb/SLAP protein and inhibit binding of an Ena/VASP protein to the Fyb/SLAP protein. For example, polypeptides which have a functional EVH1 domain to enable Fyb/SLAP binding but which cannot function in cytoskeletal rearrangement. Exemplary polypeptides of this type are nonfunctional Ena/VASP proteins such as Ena/VASP fragments, and chimeric polypeptides containing EVH1 domain without other Ena/VASP sequences.

Other Fyb/SLAP complex inhibitors include molecules which selectively reduce expression of one or more of the proteins in the Fyb/SLAP complex, such as antisense nucleic acid molecules and molecules which bind to the upstream regulatory sequences of the genes which encode the proteins in the complex and reduce the expression of the genes.

Decreasing the activity of the complexes and signaling pathways leading to cytoskeletal rearrangement is useful in reducing inappropriate or excessive immune responses (i.e., immunosuppression). For example, a subject that has or is at risk of developing an autoimmune disease or a condition characterized by inflammation could benefit from these methods.

In other embodiments, the invention provides methods for enhancing cytoskeletal rearrangement in a cell or cell fragment. Such methods are useful, for example, for increasing responses of hematopoietic cells upon stimulation. For example, T cells which do not fully activate upon T cell receptor engagement may have defects in the signaling pathway from the T cell receptor to the actin cytoskeleton. Based on the disclosure herein, one of ordinary skill in the art can determine if one or more components of the protein complexes which mediate such signaling are deficient or defective. One then can supplement the deficient or defective protein to increase the activity of the complex and restore normal signaling. Increasing the activity of the complexes and signaling pathways leading to cytoskeletal rearrangement also is useful in boosting immune responses in conditions which involve beneficial immune responses. For example, a subject that has or is at risk of developing an infectious disease or cancer could benefit from an enhanced immune response.

An example of these methods includes contacting a cell (or cell fragment) with an amount of a composition which increases the amount of a Fyb/SLAP polypeptide in the cell (or cell fragment) sufficient to enhance the formation of a complex of an Ena/VASP protein and a Fyb/SLAP protein. Exemplary compositions include Fyb/SLAP polypeptides and nucleic acid molecules which encode Fyb/SLAP polypeptides. Other members of the signaling complexes as set forth herein also can be increased in like manner to elicit a similar effect.

The modulators of the Fyb/SLAP complexes can be administered in vivo to a subject in need of such treatment. As used herein, a “subject” includes a human, non-human primate, cow, horse, pig, sheep, goat, dog, cat or rodent. In all embodiments human subjects are preferred. Any convenient mode of administration which brings the modulator in contact with the protein complex can be used.

The methods of the invention preferably are employed with hematopoietic cells, more preferably macrophages or lymphocytes, and still more preferably T cells (e.g., helper T cells or cytotoxic T cells). The methods of the invention also can be employed with cell fragments such as platelets, for example, for modulation of platelet aggregation in wound healing or clotting.

The invention involves in another aspect isolated Fyb/SLAP2 polypeptides, isolated Fyb/SLAP2 nucleic acid molecules encoding those polypeptides, functional modifications and variants of the foregoing, useful fragments of the foregoing, as well as diagnostics and therapeutics relating thereto. For uses and methods relating to modulation of hematopoietic cell activity, particularly T cell activity, the term “Fyb/SLAP” includes both forms of the Fyb/SLAP nucleic acids and polypeptides identified herein, namely Fyb/SLAP1 and Fyb/SLAP2.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulatable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulatable by standard techniques known to those of ordinary skill in the art. An isolated nucleic acid as used herein is not a naturally occurring chromosome.

As used herein with respect to polypeptides, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

Homologs and alleles of the Fyb/SLAP2 nucleic acids of the invention can be identified by conventional techniques. Thus, an aspect of the invention is those nucleic acid sequences which code for polypeptides having the insert sequence of Fyb/SLAP2, and related homologous sequences. The invention does not include homologs of Fyb/SLAP2 known as of the filing date of this application, such as murine FYB-130 (GenBank accession number AF061744).

The term “stringent hybridization conditions” and the like as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent hybridization conditions, as used herein, refers, for example, to hybridization at 65° C. in hybridization buffer (3.5× SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). SSC is 0.15M sodium chloride/0.15M sodium citrate, pH7; SDS is sodium dodecyl sulphate; and EDTA is ethylenediaminetetracetic acid. After hybridization, the membrane upon which the DNA is transferred is washed, for example, in 2× SSC at room temperature and then at 0.1-0.5× SSC/0.1× SDS at temperatures up to 68° C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of Fyb/SLAP2 nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

In general homologs and alleles typically will share at least 75% nucleotide identity and/or at least 90% amino acid identity to the sequences of Fyb/SLAP2 nucleic acid and polypeptides (particularly the insert present in Fyb/SLAP2 but absent in Fyb/SLAP1), respectively, in some instances will share at least 90% nucleotide identity and/or at least 95% amino acid identity and in still other instances will share at least 95% nucleotide identity and/or at least 99% amino acid identity. The homology can be calculated using various, publicly available software tools developed by National Center for Biotechnology Information (NCBI, Bethesda, Md.) that can be obtained through the internet from the NCBI website. Exemplary tools include the BLAST software available from the NCBI website, using default settings. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In screening for Fyb/SLAP2 nucleic acid molecules, a Southern blot may be performed using the foregoing conditions, together with a labeled (e.g., radioactive, chemiluminescent) probe. After washing the membrane to which the DNA is finally transferred, the membrane can be placed against X-ray film or a phosphorimager screen to detect the radioactive signal. In screening for the expression of Fyb/SLAP2 nucleic acids, Northern blot hybridizations using the foregoing conditions can be performed on RNA isolated from tissues or cells (e.g., T cells). Amplification protocols such as polymerase chain reaction using primers which hybridize to the sequences presented also can be used for detection of the Fyb/SLAP2 nucleic acid molecules or expression thereof.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating Fyb/SLAP polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as antigenicity, enzymatic activity, receptor binding, formation of protein complexes, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention also provides isolated unique fragments of Fyb/SLAP2 nucleic acid sequences or complements thereof. A unique fragment is one that is a ‘signature’ for the larger nucleic acid. It, for example, is long enough to assure that its precise sequence is not found in molecules within the human genome outside of the Fyb/SLAP2 nucleic acids defined above (and human alleles thereof). Those of ordinary skill in the art may apply no more than routine procedures to determine if a fragment is unique within the human genome. Unique fragments, however, exclude fragments completely composed of the nucleotide sequences of GenBank accession number AF06 1744 or other previously published sequences as of the filing date of the priority documents for sequences listed in a respective priority document or the filing date of this application for sequences listed for the first time in this application which overlap the sequences of the invention.

A fragment which is completely composed of the sequence described in the foregoing GenBank deposits is one which does not include any of the nucleotides unique to the sequences of the invention. Thus, a unique fragment must contain a nucleotide sequence other than the exact sequence of those in GenBank or fragments thereof. The difference may be an addition, deletion or substitution with respect to the GenBank sequence or it may be a sequence wholly separate from the GenBank sequence.

Unique fragments can be used as probes in Southern and Northern blot assays to identify such nucleic acids, or can be used in amplification assays such as those employing PCR. As known to those skilled in the art, large probes such as 200, 250, 300 or more nucleotides are preferred for certain uses such as Southern and Northern blots, while smaller fragments will be preferred for uses such as PCR. Unique fragments also can be used to produce fusion proteins for generating antibodies or determining binding of the polypeptide fragments, or for generating immunoassay components. Likewise, unique fragments can be employed to produce nonfused fragments of the Fyb/SLAP2 polyp eptides, useful, for example, in the preparation of antibodies, in immunoassays and in modulating protein complex formation in T cells. Unique fragments further can be used as antisense molecules to inhibit the expression of Fyb/SLAP2 nucleic acids and polypeptides, particularly for therapeutic purposes as described in greater detail below.

As will be recognized by those skilled in the art, the size of the unique fragment will depend upon its conservancy in the genetic code. Thus, some regions of Fyb/SLAP2 sequences and complements thereof will require longer segments to be unique while others will require only short segments, typically between 12 and 32 nucleotides (e.g. 12, 13, 14, 15, 16, 17, 18, 19, 20, 21, 22, 23, 24, 25, 26, 27, 28, 29, 30, 31 and 32 or more bases long, up to the entire length of the disclosed sequence. This disclosure intends to embrace each and every fragment of each sequence, beginning at the first nucleotide, the second nucleotide and so on, up to 8 nucleotides short of the end, and ending anywhere from nucleotide number 8, 9, 10 and so on for each sequence, up to the very last nucleotide (provided the sequence is unique as described above).

Virtually any segment of the polypeptide coding region of novel Fyb/SLAP2 nucleic acids, or complements thereof, that is 18 or more nucleotides in length will be unique. Those skilled in the art are well versed in methods for selecting such sequences, typically on the basis of the ability of the unique fragment to selectively distinguish the sequence of interest from other sequences in the human genome of the fragment to those on known databases typically is all that is necessary, although in vitro confirmatory hybridization and sequencing analysis may be performed.

The invention also embraces antisense oligonucleotides that selectively bind to a nucleic acid molecule encoding a Fyb/SLAP polypeptide, to reduce the expression of Fyb/SLAP sequences. This is desirable in virtually any medical condition wherein a reduction of expression of Fyb/SLAP sequences is desirable, e.g., in the treatment of inappropriate T cell activation. This is also useful for in vitro or in vivo testing of the effects of a reduction of expression of Fyb/SLAP sequences, including both Fyb/SLAP1 and Fyb/SLAP2.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions.

Based upon the sequences of nucleic acids encoding Fyb/SLAP polypeptides, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 15-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred for the purpose of reducing expression of alternative transcripts if alternative mRNA splicing occurs. Targeting to mRNA splicing sites may be preferred, however, where a reduction of one or more of these alternative transcripts is desired. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. Finally, although the sequences provided herein are cDNA sequences, one of ordinary skill in the art may easily derive the genomic DNA corresponding to the cDNA of a Fyb/SLAP polypeptide. Thus, the present invention also provides for antisense oligonucleotides which are complementary to the genomic DNA corresponding to nucleic acids encoding Fyb/SLAP polypeptides. Similarly, antisense to allelic or homologous cDNAs and genomic DNAs are enabled without undue experimentation.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic intemucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic intemucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, nucleic acids encoding Fyb/SLAP polypeptides, together with pharmaceutically acceptable carriers.

Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art, as further described below.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operablyjoined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a Fyb/SLAP polypeptide or fragment or variant thereof. That heterologous DNA (RNA) is placed under operable control of transcriptional elements to permit the expression of the heterologous DNA in the host cell.

Preferred systems for mRNA expression in mammalian cells are those such as pcDNA3.1 and pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein-Barr Virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant for the expression of an antigen is disclosed by Warnier et al., in intradermal injection in mice for immunization against P1 A (Int. J. Cancer, 67:303-310, 1996). Additional vectors for delivery of nucleic acids are known to one of ordinary skill in the art.

Various techniques may be employed for introducing nucleic acids of the invention into cells, depending on whether the nucleic acids are introduced in vitro or in vivo in a host. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

The invention also embraces so-called expression kits, which allow the artisan to prepare a desired expression vector or vectors. Such expression kits include at least separate portions of a vector and one or more of the previously discussed Fyb/SLAP nucleic acid molecules. Other components may be added, as desired, as long as the previously mentioned nucleic acid molecules, which are required, are included. The invention also includes kits for amplification of a Fyb/SLAP nucleic acid, including at least one pair of amplification primers which hybridize to a Fyb/SLAP nucleic acid. The primers preferably are 12-32 nucleotides in length and are non-overlapping to prevent formation of “primer-dimers”. One of the primers will hybridize to one strand of the Fyb/SLAP nucleic acid and the second primer will hybridize to the complementary strand of the Fyb/SLAP nucleic acid, in an arrangement which permits amplification of the Fyb/SLAP nucleic acid. Selection of appropriate primer pairs is standard in the art. For example, the selection can be made with assistance of a computer program designed for such a purpose, optionally followed by testing the primers for amplification specificity and efficiency.

The invention also permits the construction of Fyb/SLAP gene “knock-outs” in cells and in animals, providing materials for studying certain aspects of immune system responses.

The invention also provides isolated polypeptides encoded by the foregoing Fyb/SLAP nucleic acids, including whole proteins, partial proteins (e.g., domains) and peptides (e.g., EVH1 binding motif peptides). Such polypeptides are useful, for example, alone or as fusion proteins to generate antibodies, as targets for screening compounds for immunosuppressive reagents that bind Fyb/SLAP, as components of an immunoassay or diagnostic assay or as therapeutics. Fyb/SLAP polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including peptides such as the aforementioned EVH1 binding peptides also can be synthesized chemically using well-established methods of peptide synthesis.

A unique fragment of a Fyb/SLAP polypeptide, in general, has the features and characteristics of unique fragments as discussed above in connection with nucleic acids. As will be recognized by those skilled in the art, the size of the unique fragment will depend upon factors such as whether the fragment constitutes a portion of a conserved protein domain. Thus, some regions of Fyb/SLAP polypeptides will require longer segments to be unique while others will require only short segments, typically between 5 and 12 amino acids (e.g. 5, 6, 7, 8, 9, 10, 11 or 12 or more amino acids including each integer up to one less amino acid than the full length of the polypeptide).

Unique fragments of a polypeptide preferably are those fragments which retain a distinct functional capability of the polypeptide. Functional capabilities which can be retained in a unique fragment of a polypeptide include interaction with antibodies, interaction with other polypeptides or fragments thereof, selective binding of nucleic acids or proteins, and enzymatic activity. One important activity is the ability to act as a signature for identifying the polypeptide. Another activity is the ability to interfere with the formation of a protein complex and thereby alter a T cell-mediated immune response. Another activity is the ability to interfere with macrophage activation. Those skilled in the art are well versed in methods for selecting unique amino acid sequences, typically on the basis of the ability of the unique fragment to selectively distinguish the sequence of interest from non-family members. A comparison of the sequence of the fragment to those on known databases typically is all that is necessary.

The invention embraces variants of the Fyb/SLAP polypeptides described above. As used herein, a “variant” of a Fyb/SLAP polypeptide is a polypeptide which contains one or more modifications to the primary amino acid sequence of a Fyb/SLAP polypeptide. Modifications which create a Fyb/SLAP polypeptide variant can be made to a Fyb/SLAP polypeptide 1) to reduce or eliminate an activity of a Fyb/SLAP polypeptide; 2) to enhance a property of a Fyb/SLAP polypeptide, such as protein stability in an expression system or the stability of protein-protein binding; or 3) to provide a novel activity or property to a Fyb/SLAP polypeptide, such as addition of an antigenic epitope or addition of a detectable moiety (such as the green fluorescent protein (GFP) fusions exemplified below). Modifications to a Fyb/SLAP polypeptide are typically made to the nucleic acid which encodes the Fyb/SLAP polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin or GFP, addition of a fatty acid, and the like. Modifications also embrace fusion proteins comprising all or part of the Fyb/SLAP amino acid sequence.

One of skill in the art will be familiar with methods for predicting the effect on protein conformation of a change in protein sequence, and can thus “design” a variant Fyb/SLAP polypeptide according to known methods. One example of such a method is described by Dahiyat and Mayo in Science 278:82-87, 1997, whereby proteins can be designed de novo. The method can be applied to a known protein to vary only a portion of the polypeptide sequence. By applying the computational methods of Dahiyat and Mayo, specific variants of a Fyb/SLAP polypeptide can be proposed and tested to determine whether the variant retains a desired conformation.

In general, variants include Fyb/SLAP polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a Fyb/SLAP polypeptide by eliminating proteolysis by proteases in an expression system (e.g., dibasic amino acid residues in yeast expression systems in which KEX2 protease activity is present).

Mutations of a nucleic acid which encode a Fyb/SLAP polypeptide preferably preserve the amino acid reading frame of the coding sequence, and preferably do not create regions in the nucleic acid which are likely to hybridize to form secondary structures, such as hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant Fyb/SLAP polypeptides) which are silent as to the amino acid sequence of the polypeptide, but which provide preferred codons for translation in a particular host. The preferred codons for translation of a nucleic acid in, e.g., E. Coli, are well known to those of ordinary skill in the art. Still other mutations can be made to the noncoding sequences of a Fyb/SLAP gene or cDNA clone to enhance expression of the polypeptide. The activity of variants of Fyb/SLAP polypeptides can be tested by cloning the gene encoding the variant Fyb/SLAP polypeptide into a bacterial or mammalian expression vector, introducing the vector into an appropriate host cell, expressing the variant Fyb/SLAP polypeptide, and testing for a functional capability of the Fyb/SLAP polypeptides as disclosed herein. For example, the variant Fyb/SLAP polypeptide can be tested for antibody binding or protein complex formation as disclosed in the Examples. Preparation of other variant polypeptides may favor testing of other activities, as will be known to one of ordinary skill in the art.

The skilled artisan will also realize that conservative amino acid substitutions may be made in Fyb/SLAP polypeptides to provide functionally equivalent variants of the foregoing polypeptides, i.e., the variants retain the functional capabilities of the Fyb/SLAP polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the Fyb/SLAP polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a peptide derived from a Fyb/SLAP polypeptide binds to EVH1 proteins alone or in a complex (e.g., as described in the Examples), one can make conservative amino acid substitutions to the amino acid sequence of the peptide. The substituted peptides can then be tested for binding to the MHC molecule and recognition by CTLs when bound to MHC. These variants can be tested for improved stability and are useful, inter alia, in vaccine compositions.

Conservative amino-acid substitutions in the amino acid sequence of Fyb/SLAP polypeptides to produce functionally equivalent variants of Fyb/SLAP polypeptides typically are made by alteration of a nucleic acid encoding a Fyb/SLAP polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a Fyb/SLAP polypeptide. Where amino acid substitutions are made to a small unique fragment of a Fyb/SLAP polypeptide, such as an EVH1 binding peptide, the substitutions can be made by directly synthesizing the peptide. The activity of functionally equivalent fragments of Fyb/SLAP polypeptides can be tested by cloning the gene encoding the altered Fyb/SLAP polypeptide into a bacterial or mammalian expression vector, introducing the vector into an appropriate host cell, expressing the altered Fyb/SLAP polypeptide, and testing for a functional capability of the Fyb/SLAP polypeptides as disclosed herein. Peptides which are chemically synthesized can be tested directly for function, e.g., for binding to antisera recognizing associated antigens.

The invention as described herein has a number of uses, some of which are described elsewhere herein. First, the invention permits isolation of the Fyb/SLAP protein molecules. A variety of methodologies well-known to the skilled practitioner can be utilized to obtain isolated Fyb/SLAP molecules. The polypeptide may be purified from cells which naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide. Translation of mRNA in cell-free extracts such as the reticulocyte lysate system also may be used to produce polypeptide. Those skilled in the art also can readily follow known methods for isolating Fyb/SLAP polypeptides. These include, but are not limited to, immunochromatography, HPLC, size-exclusion chromatography, ion-exchange chromatography and immune-affinity chromatography.

The isolation and identification of Fyb/SLAP genes also makes it possible for the person of skill in the art to diagnose a disorder characterized by expression of Fyb/SLAP sequences. These methods involve determining expression of one or more Fyb/SLAP nucleic acids, and/or encoded Fyb/SLAP polypeptides and/or peptides derived therefrom. In the former situation, such determinations can be carried out via any standard nucleic acid determination assay, including the polymerase chain reaction, or assaying with labeled hybridization probes. In the latter situation, such determinations can be carried out, for example, by screening samples isolated from a subject with agents that selectively bind the Fyb/SLAP polypeptides, or by screening patient antisera for recognition of the polypeptide. Sequentially obtained samples also can be obtained from a patient and assayed for amounts of Fyb/SLAP nucleic acids or polypeptides for monitoring the progress of the disease or of the patient's response to therapeutic agents. Thus the methods of diagnosis can contribute to a determination of the patient's prognosis as well as determination of the regression, progression or onset of a disease or condition.

Any convenient format may be used for the diagnostic methods based on the Fyb/SLAP sequences. These include, but are not limited to, vials, microwells, chips and dipsticks preloaded with nucleic acid molecules and/or other reagents. Such components may be packaged as a kit with containers of reagents required for carrying out the diagnostic methods, optionally including instructions.

The invention also makes it possible to isolate proteins which bind to Fyb/SLAP polypeptides as disclosed herein, including antibodies and cellular binding partners of the Fyb/SLAP polypeptides. Additional uses are described further herein.

The invention also provides, in certain embodiments, “dominant negative” polypeptides derived from Fyb/SLAP polypeptides. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide in a cell is a reduction in function of active proteins. One of ordinary skill in the art can assess the potential for a dominant negative variant of a protein, and using standard mutagenesis techniques to create one or more dominant negative variant polypeptides. For example, given the teachings contained herein of Fyb/SLAP polypeptides, especially those which are similar to known proteins, one of ordinary skill in the art can modify the sequence of the Fyb/SLAP polypeptides by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. In certain embodiments, the phosphorylation sites of Fyb/SLAP can be altered to alter the activity of the protein (e.g., by interfering with its interaction with other proteins or by preventing its phosphorylation). The skilled artisan then can test the population of mutagenized polypeptides for diminution in a selected activity and/or for retention of such an activity. Other similar methods for creating and testing dominant negative variants of a protein will be apparent to one of ordinary skill in the art.

In preferred embodiments, dominant negative Fyb/SLAP proteins can be used as immunosuppressive agents, or to screen for immune response altering compounds.

The invention also involves agents such as polypeptides which bind to Fyb/SLAP polypeptides. Such binding agents can be used, for example, in screening assays to detect the presence or absence of Fyb/SLAP polypeptides and complexes of Fyb/SLAP polypeptides and their binding partners and in purification protocols to isolated Fyb/SLAP polypeptides and complexes of Fyb/SLAP polypeptides and their binding partners. Such agents also can be used to inhibit the native activity of the Fyb/SLAP polypeptides, for example, by binding to such polypeptides.

The invention, therefore, embraces peptide binding agents which, for example, can be antibodies or fragments of antibodies having the ability to selectively bind to Fyb/SLAP polypeptides. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pfc′ region has been enzymatically cleaved, or which has been produced without the pfc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Fully human monoclonal antibodies also can be prepared by immunizing mice transgenic for large portions of human immunoglobulin heavy and light chain loci. See, e.g., U.S. Pat. Nos. 5,545,806, 6,150,584, and references cited therein. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to Fyb/SLAP polypeptides, and complexes of both Fyb/SLAP polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention, including human antibodies. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the Fyb/SLAP polypeptide. This process can be repeated through several cycles of reselection of phage that bind to the Fyb/SLAP polypeptide. Repeated rounds lead to enrichment of phage bearing particular sequences. DNA sequence analysis can be conducted to identify the sequences of the expressed polypeptides. The minimal linear portion of the sequence that binds to the Fyb/SLAP polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the Fyb/SLAP polypeptides. Thus, the Fyb/SLAP polypeptides of the invention, or a fragment thereof, can be used to screen peptide libraries, including phage display libraries, to identify and select peptide binding partners of the Fyb/SLAP polypeptides of the invention. Such molecules can be used, as described, for screening assays, for purification protocols, for interfering directly with the functioning of Fyb/SLAP polypeptide and for other purposes that will be apparent to those of ordinary skill in the art.

As detailed herein, the foregoing antibodies and other binding molecules may be used for example to identify tissues expressing protein or to purify protein. Antibodies also may be coupled to specific diagnostic labeling agents for imaging of cells and tissues that express Fyb/SLAP polypeptides or to therapeutically useful agents according to standard coupling procedures. In addition, the foregoing antibodies and other binding molecules may be used to inhibit the binding of Fyb/SLAP polypeptides in protein complexes, for example, to reduce T cell responses to T cell receptor engagement.

When “disorder” is used herein, it refers to any pathological condition where the expression of Fyb/SLAP sequences are increased or decreased from normal amounts. For example, lower than normal expression of Fyb/SLAP protein could reduce the efficiency of T cell activation by reducing cytoskeletal rearrangement in response to T cell receptor engagement with an antigen presenting cell. Higher than normal expression of Fyb/SLAP protein could hyperstimulate T cells leading to inappropriate immune response such as those observed in autoimmune diseases.

Samples of tissue and/or cells for use in the various methods described herein can be obtained through standard methods of collection of blood or other bodily fluids (e.g., aspiration or other methods), or via other methods such as tissue biopsy, including punch biopsy and cell scraping.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing antibody aerosols without resort to undue experimentation. When using antisense preparations of the invention, slow intravenous administration is preferred.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The preparations of the invention are administered in effective amounts. An effective amount is that amount of a pharmaceutical preparation that alone, or together with further doses, produces the desired response. In the case of treating a condition characterized by an excessive or inappropriate immune response, such as an autoimmune disease, the desired response is inhibiting the excessive or inappropriate immune response. This may involve only slowing the progression or manifestation of the disease temporarily, although more preferably, it involves halting the progression or manifestation of the disease permanently. This can be monitored by routine methods or can be monitored according to diagnostic methods of the invention discussed herein. The effective amounts can be administered in vivo, ex vivo to cells isolated from a subject, or in vitro for diagnostic, research and testing purposes.

The invention also contemplates gene therapy. The procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy using vectors such as adenovirus, retroviruses, herpes virus, and targeted liposomes also is contemplated according to the invention.

The invention further provides efficient methods of identifying pharmacological agents or lead compounds for agents active in modulating the biological activities described herein. In particular, such activities include actin cytoskeletal rearrangement, T cell activation, macrophage activation, platelet aggregation, and Fyb/SLAP—Ena/VASP binding. Generally, the screening methods involve assaying for compounds which interfere with a Fyb/SLAP activity such as T cell activation, macrophage activation, Ena/VASP binding, etc. Such methods are adaptable to automated, high throughput screening of compounds. The target therapeutic indications for pharmacological agents detected by the screening methods are limited only in that the target cellular function be subject to modulation by Fyb/SLAP such as by alteration of the formation of a complex comprising a Fyb/SLAP polypeptide or fragment thereof and one or more natural Fyb/SLAP intracellular binding targets, such as Ena/VASP or other EVH1 domain proteins. Target indications include cellular processes modulated by actin remodeling, such as T cell activation and macrophage activation. therefore, immunosuppresive compounds can be identified by screening for compounds that interfere with Fyb/SLAP binding, Fyb/SLAP phosphorylation, or other Fyb/SLAP activities.

A wide variety of assays for pharmacological agents are provided, including, labeled in vitro protein-protein binding assays, electrophoretic mobility shift assays, immunoassays, cell-based assays such as two- or three-hybrid screens, expression assays, etc. For example, three-hybrid screens are used to rapidly examine the effect of transfected nucleic acids on the intracellular binding of Fyb/SLAP or Fyb/SLAP fragments to specific intracellular targets. The transfected nucleic acids can encode, for example, combinatorial peptide libraries or antisense molecules. Convenient reagents for such assays, e.g., GAL4 fusion proteins, are known in the art and are described further in the Examples. An exemplary cell-based assay involves transfecting a cell with a nucleic acid encoding a Ena/VASP binding Fyb/SLAP polypeptide fused to a GAL4 DNA binding domain and a nucleic acid encoding a Ena/VASP protein fused to a transcription activation domain such as VP16. The cell also contains a reporter gene operably linked to a gene expression regulatory region, such as one or more GAL4 binding sites. Activation of reporter gene transcription occurs when the Fyb/SLAP and Ena/VASP polypeptides bind such that the GAL4 DNA binding domain and the VP 16 transcriptional activation domain are brought into proximity to enable transcription of the reporter gene. Agents which modulate a Fyb/SLAP polypeptide mediated cell function are then detected through a change in the expression of reporter gene. Methods for determining changes in the expression of a reporter gene are known in the art.

Fyb/SLAP fragments used in the methods, when not produced by a transfected nucleic acid are added to an assay mixture as an isolated polypeptide. Fyb/SLAP polypeptides preferably are produced recombinantly, although such polypeptides may be isolated from biological extracts. Recombinantly produced Fyb/SLAP polypeptides include chimeric proteins comprising a fusion of a Fyb/SLAP protein with another polypeptide, e.g., a polypeptide capable of providing or enhancing protein-protein binding, sequence specific nucleic acid binding (such as GAL4), enhancing stability of the Fyb/SLAP polypeptide under assay conditions, or providing a detectable moiety, such as green fluorescent protein. A polypeptide fused to a Fyb/SLAP polypeptide or fragment may also provide means of readily detecting the fusion protein, e.g., by immunological recognition or by fluorescent labeling.

The assay mixture is comprised of a natural intracellular Fyb/SLAP binding target such as an Ena/VASP protein or fragment thereof capable of binding to Fyb/SLAP. While natural Fyb/SLAP binding targets may be used, it is frequently preferred to use portions (e.g., peptides or nucleic acid fragments) or analogs (i.e., agents which mimic the Fyb/SLAP binding properties of the natural binding target for purposes of the assay) of the Fyb/SLAP binding target so long as the portion or analog provides binding affinity and avidity to the Fyb/SLAP fragment measurable in the assay.

The assay mixture also comprises a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with polypeptides and/or nucleic acids, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid, the agent typically is a DNA or RNA molecule, although modified nucleic acids as defined herein are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, acidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease, inhibitors, nuclease inhibitors, antimicrobial agents, and the like may also be used.

The mixture of the foregoing assay materials is incubated under conditions whereby, but for the presence of the candidate pharmacological agent, the Fyb/SLAP polypeptide specifically binds the cellular binding target, a portion thereof or analog thereof. The order of addition of components, incubation temperature, time of incubation, and other perimeters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of specific binding between the Fyb/SLAP polypeptide and one or more binding targets is detected by any convenient method available to the user. For cell free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as two- or three-hybrid screens. The transcript resulting from a reporter gene transcription assay of Fyb/SLAP polypeptide binding to a target molecule typically encodes a directly or indirectly detectable product, e.g., β-galactosidase activity, luciferase activity, and the like. For cell free binding assays, one of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseradish peroxidase, etc.). The label may be bound to a Fyb/SLAP binding partner, or incorporated into the structure of the binding partner.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

The invention provides Fyb/SLAP-specific binding agents, methods of identifying and making such agents, and their use in diagnosis, therapy and pharmaceutical development. For example, Fyb/SLAP-specific pharmacological agents are useful in a variety of diagnostic and therapeutic applications, especially where disease or disease prognosis is associated with improper utilization of a pathway involving Fyb/SLAP, e.g., Fyb/SLAP-Ena/VASP complex formation, etc. In particular, immunosuppressive agents that interfere with Fyb/SLAP function are contemplated and provided. Novel Fyb/SLAP-specific binding agents include Fyb/SLAP-specific antibodies and other natural intracellular binding agents identified with assays such as two hybrid screens, and non-natural intracellular binding agents identified in screens of chemical libraries and the like.

In general, the specificity of Fyb/SLAP binding to a binding agent is shown by binding equilibrium constants. Targets which are capable of selectively binding a Fyb/SLAP polypeptide preferably have binding equilibrium constants of at least about 10⁷ M⁻¹, more preferably at least about 10⁸ M⁻¹, and most preferably at least about 10⁹ M⁻¹. The wide variety of cell based and cell free assays may be used to demonstrate Fyb/SLAP P4-specific binding. Cell based assays include one, two and three hybrid screens, assays in which Fyb/SLAP-mediated transcription is inhibited or increased, etc. Cell free assays include Fyb/SLAP-protein binding assays, immunoassays, etc. Other assays useful for screening agents which bind Fyb/SLAP polypeptides include fluorescence resonance energy transfer (FRET), and electrophoretic mobility shift analysis (EMSA).

### EXAMPLES

### Example 1

Introduction

To identify novel EVH1-binding proteins, a mouse embryonic expression library was screened with polyclonal antibodies raised against the EVH1-binding motif of ActA that had been previously observed to react with vinculin, zyxin, and several unidentified bands on western blots of HeLa cell lysates (Niebuhr et al., 1997). Positive clones were identified and one, clone 5/7, which bound to the [³²P]-labeled GST-EVH1 domain of Mena in a blot overlay assay, was subsequently screened against the Expressed Sequence Tag (EST) database and an unknown protein encoded by a 2.3-kb cDNA was identified and characterized.

Methods

Screening of a mouse embryo (d16) expression library λExlox; Novagen Inc., Madison, Wis.) was carried out using polyclonal antibodies raised against the synthetic peptide SFEFPPPPTDEELRL (SEQ ID NO:1) derived from ActA (Niebuhr et al., 1997). The EST clone (IMAGE clone ID 221953; RZPD IMAGp998F02441) was obtained from the Resource Centre of the German Human Genome Project (RZPD), Berlin, Germany. Complete RNA was purified from HL60 cells using Trizol reagent (Life Technologies, Inc. Rockville, Md.) according to the manufacturer's instructions. RT-PCR was carried out using the “First strand synthesis kit” according to the provided instructions (Amersham Pharmacia Biotech, Inc. Piscataway, N.J.). Sequencing and polymerase chain reactions were performed according to standard procedures.

Results

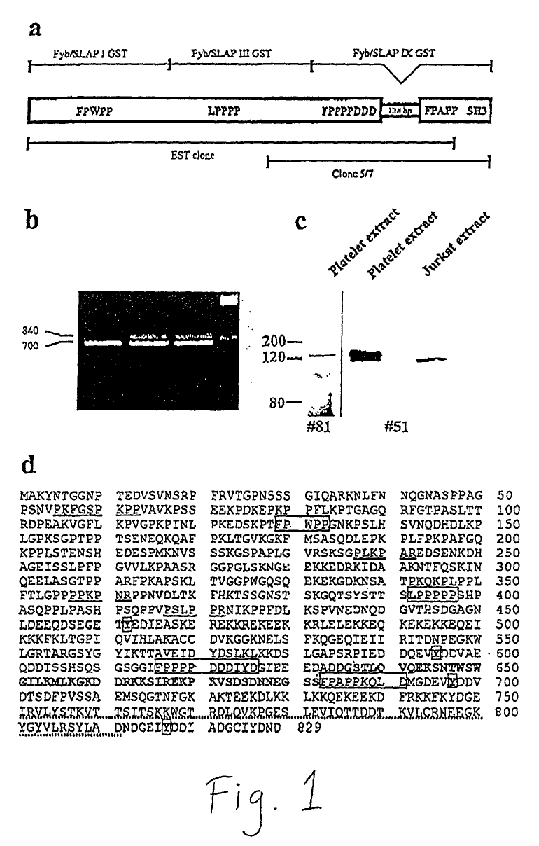

Screening the mouse embryonic expression library identified 15 positive clones. By screening the Expressed Sequence Tag (EST) database with the sequence of one of the positive clones (5/7), a partial clone, revealed a human EST clone (IMAGE clone ID 221953; RZPD IMAGp998F02441) that contained a 2.3 kb cDNA encoding an unknown protein, which is illustrated in FIG. 1a. During the characterization of this protein, two identical protein sequences, termed Fyb (Da Silva et al., 1997) or SLAP-130 (Musci et al., 1997), were reported. The EST-clone was identical to the published sequence of Fyb/SLAP except for an additional insertion of 131 bp and a frame shift that leads to an earlier termination of translation. Three independent RT-PCRs of the carboxy-terminus of Fyb/SLAP showed two bands of 700 and 840 bp, illustrated in FIG. 1b, indicating that two isoforms of Fyb/SLAP, at least in the human myeloid cell line HL60, exist. The sequences of these fragments indicated that the longer isoform contains an insertion of 138 bp and has the same carboxy-terminus as Fyb/SLAP. The 700-bp fragment corresponds to the published sequence. A full-length Fyb/SLAP clone without the 138-bp insertion was obtained by RT-PCR and termed Fyb/SLAPI (SEQ ID NOs:2 and 3; SEQ ID NO:3 is the nucleotide sequence encoding SEQ ID NO:2). The full-length cDNA clone of the longer Fyb/SLAP isoform was amplified by PCR using the EST clone and the 840-bp RT-PCR fragment as templates, and was termed Fyb/SLAP2 (SEQ ID NOs:4 and 5; SEQ ID NO:5 is the nucleotide sequence encoding SEQ ID NO:4) (FIG. 1a). The murine homolog of Fyb/SLAP2, also known as FYB-130 (SEQ ID NO:14), has also been cloned (Veale et al., 1999). Its structure is identical to murine FYB-120 with the addition of a 46 amino acid insertion in the protein's carboxyl-terminal region.

### Example 2

Introduction

The Fyb/SLAP sequence was analyzed to identify its domain structure. In addition, to investigate Fyb/SLAP in more detail, a polyclonal antiserum (#51) was raised against, and affinity purified with, a recombinant Fyb/SLAP fragment covering the central domain common to both isoforms (Fyb/SLAP III GST, FIG. 1A). An additional antiserum (#81) was raised against a synthetic peptide derived from the unique sequence of Fyb/SLAP2. Both antisera were used to assess Fyb/SLAP in more detail in human platelet extracts and immunoblots of human platelet extracts.

Methods

Fusion Proteins and Antibody Production

Fragments encoding amino acids 1-339 (Fyb/SLAP I), 341-598 (Fyb/SLAP III), 548-783 (Fyb/SLAP IX) of Fyb/SLAP1 (SEQ ID NO:2) (numbering according to Da Silva et al., 1997; Musci et al., 1997), and the amino-terminus of murine WASP (amino acids 1-256; SEQ ID NO:6 Derry et al., 1995) were generated by PCR and cloned into the pGEX-2TK, pGEX-6P1 or pGEX-5×3 vectors (Amersham Pharmacia Biotech). Fusion proteins were purified on Glutathion-sepharose (Amersham Pharmacia Biotech) or Glutathion-agarose (Pierce Chemical Company, Rockford, Ill.). Immobilized Fyb/SLAP III GST was digested with thrombin (Amersham Pharmacia Biotech) and used to raise the polyclonal rabbit antiserum #51 (Eurogentec Bel S.A.), which was affinity purified using Fyb/SLAP III GST sepharose. The antiserum #81 was raised against the synthetic peptide 656-LKGKDDRKKSIREKPKV-672 (SEQ ID NO:7) derived from Fyb/SLAP2 (SEQ ID NO:4, FIG. 1D) and affinity purified using the same peptide immobilized on EAH-sepharose (Amersham Pharmacia Biotech). The proteins Fyb/SLAP I, III, IX GST prepared from pGEX-6P1 constructs, digested with PreScission protease (Amersham Pharmacia Biotech), and GST-WASP were used to raise monoclonal antibodies as previously described (Niebuhr et al., 1998).

Cell Extract Preparation and Immunoblots

Human platelet extracts were prepared with modified 2X RIPA buffer (50 mM Tris-Cl, 300 mM NaCl, 2% (weight/vol) sodium deoxycholate, 2% (vol/vol) Triton-X100, 2% (weight/vol) sodium dodecylsulfate (SDS), 2 mM EDTA, pH 7.4) using 3×10⁷ platelets/ml by incubating the lysate for 30 min on ice. The lysate was then centrifuged 20 min with 16,000× g at 4° C. and the supernatant was used.

T cell extracts were prepared in ice-cold RIPA buffer (25 mM Tris-HCl pH 7.5, 150 mM NaCl, 1% NP-40, 0.5% deoxycholate and 0.1% SDS) supplemented with 1 mM phenylmethanesulfonyl fluoride (PMSF) (see also Example 3).

Immunoblots were produced according to standard procedures (Harlow, e. and Lane, D., Using Antibodies, A Laboratory Manual. (1999) Cold Spring Harbor Laboratory Press, Cold Spring Harbor, N.Y.

Results

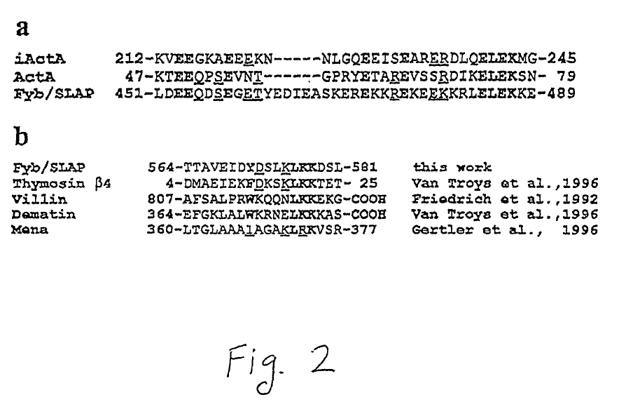

The core sequence of the EVH 1-binding site was recently characterized as F/L/WPPPP (SEQ ID NO:8; Niebuhr et al., 1997). Fyb/SLAP harbors four similar motifs (amino acids: 129-133, 392-397, 616-626, and 683-691 of SEQ ID NO:2; FIG. 1A, FIG. 1D). However, only two of them contain the adjacent acidic residues that are essential for binding to EVH1 domains as previously shown for the analogous motifs of ActA (Niebuhr et al., 1997; Carl et al., 1999). Two highly charged regions are also present within Fyb/SLAP (FIG. 1D; amino acids 451-500; amino acids 673-700 of SEQ ID NO: 2). Interestingly, the first highly charged sequence shows a striking similarity with part of the amino-terminal sequence of the L. monocytogenes protein ActA, which is involved in the actin recruitment by Listeria (FIG. 2A) (Pistor et al., 1995; Lasa et al., 1995 and 1997). The region following the first highly charged region of Fyb/SLAP (amino acids 566-581 of SEQ ID NO: 2) shows high homology with the known G-actin binding site of thymosin β4 (Van Troys et al., 1996), the human villin headpiece (Arpin et al., 1988), the putative G-actin binding site of the dematin headpiece (Van Troys et al., 1996), and a potential G-actin binding site of Ena/VASP proteins (Gertler at al., 1996) (FIG. 2B). The 46 amino acid insertion in Fyb/SLAP2 (amino acids 637-682 SEQ ID NO: 4) is highly charged and shows no homology with known motifs. The carboxy-terminus harbors an SH3-like domain (FIG. 1D) (Da Silva et al., 1997; Musci et al., 1997).

The polyclonal antiserum (#51) specifically reacted with a 120-kDa band in Jurkat T-cell extracts and with two bands of 120 and 130 kDa in extracts of human platelets (FIG. 1C). Moreover, this antiserum did not react with extracts of non hematopoietic cell lines such as HeLa. Antiserum (#81) was incubated with immunoblots of human platelet extracts and these polyclonal antibodies reacted with a 130-kDa band (FIG. 1C), indicating that the doublet detected with the antiserum #51 is indeed due to the insertion present in the Fyb/SLAP2 isoform.

### Example 3

Introduction

Because Fyb/SLAP harbors four potential EVH1-binding sites, a ligand overlay assay was utilized to determine which of these binding sites interacts with VASP and Mena. In addition, to determine if Fyb/SLAP and VASP associate in vivo, immunoprecipitates of VASP were prepared from lysates of activated human Jurkat T-cells and binding assays were performed with Fyb/SLAP.

Methods

Cell Extract Preparation

For the binding assays, Jurkat E6-1 cells were stimulated for Imin with 0.2 mM Na₃VO₄, 8 mM H₂O₂ (pervanadate), which mimics the effect of TCR ligation in Jurkat cells (Secrist et al., 1993; Motto et al., 1994). Jurkat RIPA lysates were prepared in ice-cold RIPA buffer (25 mM Tris-HCl pH 7.5, 150 mM NaCl, 1% NP-40, 0.5% deoxycholate and 0.1% SDS) supplemented with 1 mM phenylmethanesulfonyl fluoride (PMSF). Immunoprecipitations were carried out according to standard protocols using the VASP monoclonal antibody IE226C2 (Abel et al., 1996) and the WASP antiserum Fus3 (Symons et al., 1996). As negative controls, anti-ActA monoclonal antibodies (358C1) (Niebuhr et al., 1993) and a rabbit non-immune serum were used. For immunoprecipitation 0.2 ml of Jurkat RIPA lysate (derived from 1.6×10⁷ cells) was used.

Protein-overlay Assay Preparation and Use

[³⁵S]-labeled full-length VASP and the 80-kDa isoform of Mena were prepared in vitro using the TNT coupled transcription/translation system; (Promega Biosciences Inc., San Luis Obispo, Calif.). The cDNA of the ActA repeats including amino acids 241-423 of SEQ ID NO:9; (numbering as in Domann et al., 1992) was amplified by colony PCR from L. monocytogenes EGD and cloned into the pGEX-6P1 vector (Amersham Pharmacia Biotech). The murine clone 5/7 was cloned in the pGEX-6P1 vector (Amersham Pharmacia Biotech). Protein overlay assays were done as previously described (Chakraborty et al., 1995; Niebuhr et al., 1997). Spot-synthesis was performed according to Frank (1992). Membranes were analyzed using a phosphorimager (Amersham Pharmacia Biotech Inc.—Molecular Dynamics Division, Piscataway, N.J.).

Results

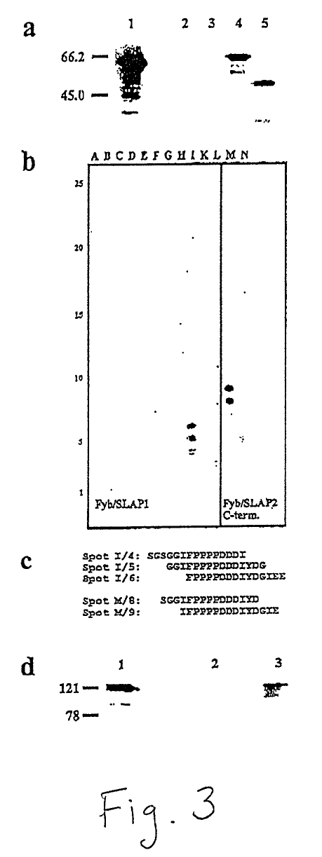

The carboxy-terminus of Fyb/SLAP (Fyb/SLAP IX GST, FIG. 1A), which harbors two potential binding sites, interacted with in vitro translated [³⁵S]-labeled VASP, whereas the central part of the protein (Fyb/SLAP III GST, FIG. 1a) and its amino-terminus (Fyb/SLAP I GST, FIG. 1A) did not (FIG. 3A). The same results were obtained using in vitro translated [³⁵S]-labeled Mena. Thus, the carboxy-terminus of Fyb/SLAP interacts directly with VASP and Mena in vitro. To map the binding site more precisely, a ligand overlay assay was performed on scans of arrayed peptides covering the complete Fyb/SLAP1 sequence and the carboxy-terminus of Fyb/SLAP2 (FIG. 3B) as recently described for the identification of the EVH1-binding sites of ActA (Niebuhr et al., 1997). As shown in FIG. 3C, the binding site for the EVH1 domain corresponds to the motif 616-FPPPPDDDI-624 (SEQ ID NO:10; FIG. 1D). The VASP immunoprecipitates contained Fyb/SLAP (FIG. 3D). These data indicate that endogenous VASP and Fyb/SLAP are present in a protein complex within hematopoietic cells.

### Example 4

Introduction

EVH1-binding motifs have been found in the cytoskeletal proteins zyxin and vinculin as well as in the Listeria protein ActA (Brindle et al., 1996; Reinhard et al., 1996; Niebuhr et al., 1997). These motifs are required for the proper subcellular targeting of Ena/VASP family proteins. Therefore, the distribution of Fyb/SLAP in platelets and activated T cells was analyzed. Platelets were allowed to attach and spread on glass coverslips, fixed and double stained for Fyb/SLAP and F-actin, VASP, zyxin, or vinculin. T-cells were double stained for F-actin and Fyb/SLAP, Ev1, WASP, Arp3, Vav, vinculin, or zyxin.

Methods

Cell Culture

Platelets were obtained from the local blood bank, diluted in lOmM HEPES pH 7.4, 5 mM KCl, 145 mM NaCl, 10 mM Glucose, 1 mM MgCl₂, 1 mM CaCl₂ and placed on glass coverslips. Platelets were allowed to settle and spread for 30 minutes in a humid-chamber at 37° C. Jurkat E6-1 (ATCC TIB 152) were grown in RPMI 1640 (Life Technologies), supplemented with 10% fetal calf serum (FCS) and 2 mM L-glutamine (Life Technologies).

Bead Assay Preparation and Use

Goat anti-mouse IgG dynabeads M-450 (Dynal, Inc. Lake Success, N.Y.) were coated with anti-CD3 monoclonal antibodies TR66 (Lanzavecchia and Scheidegger, 1987). The TR66 anti-CD3 monoclonal antibodies were a kind gift of Dr. S. Weiss (Gesellschaft für Biotechnologische Forschung, Braunschweig, Germany). Coated beads and Jurkat cells were washed with and resuspended in DMEM-HEPES (Life Technologies) supplemented with 1% fetal calf serum. They were then mixed and incubated on ice 30 min followed by 4 min at 37° C. Cells were allowed to attach on poly-L-lysine coated coverslips for 2 min on ice, fixed with 3% (w/v) paraformaldehyde in cytoskeleton buffer, pH 7.0 (CB; 10 mM PIPES, 150 mM NaCl, 5 mM EGTA, 5 mM MgCl₂, 5 mM glucose; Mies et al., 1998) and permeabilized with 0.1% (v/v) Triton-X-100 in CB pH 7.0. The zone of contact between Jurkat T-cells and anti CD3-coated beads was evaluated by measuring the length of the circular arc of contact according to the formula α*(π/180)*r, where α is the angle of the circular sector (in degrees) and r is the radius of the bead (in μm). The radius of the bead is 2.25 μm.

Immunostaining, Fixation, and Immunofluorescence Microscopy

Fixation and immunofluorescence microscopy were done as described in May et al., 1999, using the following antibodies: Fyb/SLAP #51(see Methods Section, Example 1) and 155E8, vinculin hvin1 (Sigma-Aldrich, St. Louis, Mo.), VASP IE226C2 (Abel et al., 1996), WASP (Fus3; Symons et al., 1996), Vav (mab Vav-30; Sattler et al. 1995), Ev1 (mab 84H1; Lanier et al., 1999), zyxin 164D4 (Krause et al., manuscript in preparation). The Arp3 polyclonal antibody B71 was raised against the synthetic peptide 342-(C)TVDARLKLSEELSGGRLKPK-361 (SEQ ID NO: 11) derived from bovine Arp3 sequence (GenBank accession number P32391) and affinity purified using the same peptide immobilized on EAH-sepharose (Amersham Pharmacia Biotech). CY3-phalloidin was a kind gift of Dr. H. Faulstich (Heidelberg, Germany). Fluorescently labeled secondary antibodies were purchased from Dianova (Hamburg, Germany). Image analysis was carried out as described in May et al., 1999.

Results

In glass-activated spreading human platelets Fyb/SLAP localized exclusively to lamellipodia where it overlapped with VASP, which, in addition, was also detectable in focal adhesions as previously shown (Reinhard et al., 1992). In contrast to Fyb/SLAP, zyxin (for references see Beckerle, 1997) and vinculin (for references see Jockusch et al., 1995) were predominantly detected in focal contacts, although vinculin was also found in lamellipodia as previously described (Takubo et al., 1998). The same distribution of Fyb/SLAP in spreading platelets was also obtained using the monoclonal antibody 155E8 raised against Fyb/SLAP1.

Jurkat T-cells become polarized towards anti CD3-coated beads and reorganize their actin cytoskeleton as a dense F-actin collar. Furthermore, F-actin-rich pseudopods protrude from the T-cell surface to increase the contact area at the beads/T-cells attachment sites. This approach mimics the actin reorganization taking place during T-cell/APC interaction and permits the precise analysis of typical actin remodeling of T-cells (Lowin-Kropf et al., 1998). Jurkat T-cells that bound to one bead were analyzed. In addition, only T-cell/bead conjugates in which both the T-cell and the bead were in the same plane of focus were considered suitable for analysis. This permitted the unambiguous identification of the actin collar and the achievement of consistent results from different bead assays. Interestingly, Fyb/SLAP accumulated at the T-cell/beads attachment sites where it colocalized with F-actin. Because Ev1 is the member of the Ena/VASP family proteins that is most enriched in hematopoietic cells (Lanier et al., 1999), its localization in T-cells was considered as representative of the Ena/VASP proteins in general. Ev1 localized to the contact sites between T-cells and beads. In addition, WASP, the Arp2/3 complex, e.g. Arp3, and Vav also localized at the T-cell/bead interface, whereas vinculin and zyxin did not accumulate at these contact sites.

### Example 5

Introduction

T-cells were transfected with constructs containing wild-type and mutant versions of the EVH1 domain binding sites of ActA in order to determine whether recruitment of Ena/VASP proteins is required for actin remodeling of activated T-cells. Disrupting the interaction between EVH1 domain and its ligands would likely neutralize the function of Ena/VASP proteins by preventing their proper subcellular targeting and association with natural ligands. The affinities of the Ena/VASP family proteins for the EVH1-binding sites of ActA are higher than those for their natural ligands (Niebuhr et al., 1997). Microinjection of a peptide containing an ActA EVH1-binding motif (FEFPPPPTDEELRL) (SEQ ID NO: 12) displaces Ena/VASP proteins from the focal adhesions, whereas a mutated peptide (FEAPPPPTDEELRL) (SEQ ID NO:13) has no effect (Pistor et al., 1995; Gertler et al., 1996; Niebuhr et al., 1997). To overcome the difficulty in microinjecting T-cells, and to allow a rapid identification of transfected cells, the four EVH1-binding sites of ActA (ActA-Repeats) were tagged with the Green Fluorescent Protein (GFP) (GFP-ActA-Repeats). As control, a GFP-tagged mutated variant of the four EVH1 domain-binding sites, in which the phenylalanine residues preceding the four prolines of each repeat have been mutated to alanine, was also used (GFP-ActA-Repeats F>A). In addition, the distribution of Ev1, Fyb/SLAP, WASP and the Arp2/3 complex upon the transfection described above was also analyzed in stimulated T-cells.

Methods

For descriptions of bead assay, immunostaining, cell culture, and fluorescence microscopy see Methods Section of Example 4.

GFP-constructs Production

The cDNA of the ActA repeats (amino acids 241-423 of SEQ ID NO:9; numbering as in Domann et al., 1992) were amplified by colony PCR from L. monocytogenes EGD serotype 1/2a (Domann et al., 1992). The cDNA of the mutated ActA repeats was kindly provided by Dr. Susanne Pistor (GBF, Braunschweig, Germany). The wild-type and mutant repeats were tagged with pEGFP-N1 (Clontech Laboratories Inc., Palo Alto, Calif.). As a control, a GFP-tagged mutated variant of the four EVH1 domain-binding sites, in which the phenylalanine residues preceding the four prolines of each repeat have been mutated to alanine, was used (GFP-ActA-Repeats F>A). GFP-ActA-Repeats F>A was generated by site directed mutagenesis with the QuickChange Site Directed Mutagenesis Kit (Strategene, La Jolla, Calif.) using the wild type ActA Repeats cDNA as template to introduce the appropriate mutations according to the instructions provided by the manufacturer. This construct was provided by Dr. Susanne Pistor (GBF, Braunschweig, Germany). As a positive control for this approach, HeLa cells were transfected with GFP-ActA-Repeats.

Transfection Procedure

Transfection in HeLa cells was performed with FuGENE (Roche Diagnostics, Basel, Switzerland) according to manufacturer's instructions. Jurkat E6-1 cells (1×10⁷ cells resuspended in DMEM/HEPES) were transfected with 40 82 g of cDNA by electroporation using a Gene Pulser II (Bio-Rad Laboratories, Inc. Hercules, Calif.) with the following settings: voltage: 0.25 kV, capacitance: 950 μF.

Scanning Electron Microscopy

For scanning electron microscopy (SEM), Jurkat T-cells were transfected with GFP-ActA-Repeats or GFP-ActA-Repeats F>A and sorted out using a FACS sorter (FACS Vantage, Becton Dickinson, Franklin Lakes, N.J.). This approach was necessary to obtain a cell population expressing homogeneous levels of GFP-tagged proteins and facilitated the analysis of the cells by SEM. Jurkat T-cells expressing moderate to high levels of GFP-ActA-Repeats or GFP-ActA-Repeats F>A were used for analysis. After sorting, the cells were returned to a 37° C. incubator and allowed to recover overnight. After the bead assay, Jurkat T-cells were fixed on ice 30 min with 3% PFA in CB pH 7.0, then washed with the CB buffer. Cells were postfixed with 2.5% glutaraldehyde in cacodilate buffer (0.1M sodium cacodilate, 0.09M sucrose, 10 mM MgCl₂, 10 mM CaCl₂, pH 7.2) for 2 hr. at room temperature. After washing with cacodilate buffer, the cells were dehydrated through a graded series of ethanol, processed by critical-point drying and gold coated (according to standard methods). Samples were examined with a digital scanning microscope (DSM 982 Gemini, Carl Zeiss, Inc. Thornwood, N.Y.) using a working distance of 2-4 mm and acceleration voltage of 5 Kv. Electron microscopy images were digitally handled using Photoshop 5.0 (Adobe Systems, Inc. San Jose, Calif.).

Results

Focal adhesions in the GFP-ActA-Repeats-expressing HeLa cells were obviously devoid of VASP, whereas cells transfected with GFP-ActA-Repeat F>A showed typical robust labeling of focal adhesions with VASP. When GFP-ActA-Repeats were introduced into Jurkat T-cells to test the effect of displacing Ena/VASP proteins on TCR-driven actin rearrangement, Jurkat T-cells expressing the GFP-ActA-Repeats bound to anti CD3-coated beads but did not form the typical actin collar at the T-cell/bead interface. In control untransfected cells the length of the contact zone was 5.38 μm (SD=0.98 μm; n=32) whereas in T-cells transfected with the GFP-ActA-Repeats the zone of contact was significantly reduced (3.84±0.95 μm; n=46). As a consequence of the impaired actin remodeling the F-actin accumulation was undetectable at this contact site. In contrast, cells expressing the GFP-ActA-Repeats F>A exhibited normal F-actin accumulation as well as length of the contact zone (5.16±1.33 (m; n=30) at the T-cell/bead interface. Scanning electron microscopy analysis showed that T-cells expressing GFP-ActA-Repeats simply contacted but did not surround CD3-coated beads, whereas control T-cells or cells expressing GFP-ActA-Repeats F>A almost completely surrounded the beads with sheet-like extensions. Therefore, it can be concluded that the proteins of the Ena/VASP family are essential for actin remodeling upon T-cell stimulation.

The distribution of Ev1, Fyb/SLAP, WASP and the Arp2/3 complex was also analyzed in stimulated T-cells. In Jurkat T-cells transfected with the GFP-ActA-Repeats, Ev1 did not localize at the T-cell/bead interface, whereas the distribution of Fyb/SLAP, WASP and the Arp2/3 complex was unaffected. The localization of these proteins at the T-cell/bead interface was unaffected in T-cells transfected with GFP-ActA-Repeats F>A.

### Example 6

Introduction

Based on the observation that WASP and the Arp2/3 complex localize to the attachment sites between T-cells and anti CD3-coated beads, and given that WASP family proteins directly interact with the Arp2/3 complex, it was predicted that the Arp2/3 complex is involved in actin dynamics in T-cells. The carboxy-terminal domain of Scar, ScarWA, is similar to the carboxy-terminal region of WASP and disrupts the binding of WASP family proteins to the Arp2/3 complex when expressed in mammalian cells (Machesky and Insall, 1998). Overexpression of ScarWA (which binds to G-actin and the p21 Arc subunit of the Arp2/3 complex) delocalizes the Arp2/3 complex and causes the loss of lamellipodia and stress fibers in Swiss3T3 fibroblasts and J774 macrophages. In addition, ScarWA inhibits the Listeria-induced actin tail formation in infected cells as well as in mouse brain extract. In contrast, ScarW (which binds to G-actin only) has no effect (Machesky and Insall, 1998; May et al., 1999). To investigate the role of the Arp2/3 complex in the reorganization of the T-cell actin cytoskeleton in response to TCR engagement, T-cells were transfected with myc-tagged ScarWA and ScarW, and the distribution of the Arp2/3 complex was analyzed.

Methods

For descriptions of bead assay, cell culture, general immunostaining, and fluorescence microscopy see Methods Section of Example 4.

Constructs and Transfection Procedures

The myc-tagged ScarW and ScarWA were kindly provided by Dr. Laura M. Machesky (University of Birmingham, England). Jurkat E6-1 cells (1×10⁷ cells resuspended in DMEM/HEPES) were transfected with 40 μg of cDNA by electroporation using a Gene Pulser II (Bio-Rad Laboratories, Inc) with the following settings: voltage: 0.25 kV, capacitance: 950 μF.

Myc-tag Immunostaining

The monoclonal antibody 9E10 against the myc tag was purchased from ATCC (American Type Culture Collection; clone no. CRL-1729). Myc-tag immunostaining was performed according to May et al., 1999.

Results

In Jurkat T-cells transfected with ScarWA, F-actin accumulation at the T-cell/bead interface was not detectable, whereas it was not affected in Jurkat T-cells transfected with ScarW. Moreover, the length of the contact zone between the beads and Jurkat T-cells was significantly reduced (3.16±0.78 μm; n=39) whereas in ScarW-transfected cells the size of the contact zone was comparable to that of control cells (5.00±1.08 μm; n=22). The expression of ScarWA affected the distribution of the Arp2/3 complex which was no longer detectable at the T-cell/bead interface, whereas Ev1, Fyb/SLAP and WASP still accumulated at this site. As expected, the expression of ScarW did not affect the localization of these proteins. These data indicate that, in addition to the Ena/VASP proteins, the Arp2/3 complex is essential for the reorganization of the actin cytoskeleton taking place during T-cell activation.

### Example 7

Introduction

Because Fyb/SLAP and WASP localized to the contact site between activated Jurkat T-cells and anti CD3-coated beads, both proteins might be constituents of a complex in activated Jurkat T-cells. This possibility is consistent with recent findings indicating that Fyb/SLAP binds to the SH2 domain of SLP-76 (Da Silva et al., 1997; Musci et al., 1997), which forms a ternary complex with Vav and Nck (Bubeck Wardenburg et al., 1998). In addition, Rivero-Lezcano et al. (1995) show that the SH3 domain of Nck binds to WASP. To test this hypothesis immunoprecipitates from extracts of activated Jurkat T-cells using an antiserum against WASP (Fus3, kindly provided by Dr. J. Derry, Immunex Corp., Seattle, WA) were prepared. Western blot analysis demonstrated that the immunoprecipitates contained Fyb/SLAP indicating that it is present in a protein complex with WASP in vivo.

To further investigate the composition of this complex, immunoprecipitates were prepared from Jurkat T-cell extracts using the WASP monoclonal antibody 67B4. This antibody proved to be more suitable than the polyclonal to WASP for immunoprecipitation studies, and therefore, was used to prepare affinity columns. Immunoprecipitates prepared from anti-CD3-stimulated Jurkat T-cells were analyzed using antibodies to Fyb/SLAP, SLP-76, and Nck.

In addition, because tyrosine phosphorylation is a key step during T-cell activation and least two of the proteins involved in the complex, Fyb/SLAP and SLP-76, are tyrosine phosphorylated in response to T-cell stimulation (Elements et al., 1999), the tyrosine phosphorylation pattern of WASP immunoprecipitates from Jurkat T-cells was also analyzed.

Methods

Immunoprecipitate Production

For dissection of the complex Jurkat E6-1 cells were starved overnight in RPMI 1640 (Life Technologies), supplemented with 1% (v/v) fetal calf serum (FCS) and 2 mM L-glutamine (Life Technologies). The next day the Jurkat cells were washed two times with ice-cold RPMI 1640 media without supplements and resuspended at 5×10⁷ cells/ml in ice-cold DMEM/HEPES (Life Technologies). OKT3 anti-CD3 antibodies (ATCC# CRL-8001) were added to a final concentration of 3 μg/ml for stimulation or no antibodies as control and cells were kept on ice for 15 min. After quick pelleting, stimulated and control cells were resuspended and incubated in DMEM/HEPES containing 15 λg/ml goat anti mouse IgG antibodies at 37° C. for 2 min. Jurkat NP-40 lysates were prepared at 1×10⁸ cells/ml in ice cold NP-40 lysis buffer (1% (v/v) NP-40, 150 mm NaCl, 10 mM Tris-Cl pH 7.8) supplemented with 60 μg/ml chymostatin, 10 g/ml pepstatin, 5 μg/ml leupeptin, 2 μg/ml aprotinin, 2 mM Pefabloc (Roche Diagnostics), 1 mM Na₃VO₄, and 10 mM NaF. For immunoprecipitations, affinity columns were prepared with the anti WASP 67B4 and, as control, with the anti ActA 358C1 monoclonal antibodies (Niebuhr et al., 1993) using CNBr-sepharose (Amersham Pharmacia Biotech). Immunoprecipitations were carried out according to standard protocols using 0.2 ml of Jurkat NP-40 lysates derived from 2×107 cells.

Immunoprecipitates Use

Immunoprecipitates were resolved by SDS-PAGE. As positive control, Jurkat E6-1 lysates were used. Blots were probed with antibodies to: Fyb/SLAP #51, Nck (BD Transduction Laboratories Lexington, Ky., ), SLP-76 (BD Transduction Laboratories), zyxin 164D4 (Krause et al., manuscript in preparation, and phosphotyrosine 4G10 (Upstate Biotechnology, Inc. Lake Placid, N.Y.) and processed using ECL or ECL+ enhanced chemiluminescence detection kits (Amersham Pharmacia Biotech).

Results

Fyb/SLAP, SLP-76, and Nck co-immunoprecipitated with WASP indicating that these proteins form a multiprotein complex. To rule out any nonspecific immunoprecipitation, the same immunoprecipitates were probed with the monoclonal antibody to zyxin, which does not localize at the interface between Jurkat T-cells and beads. As expected, zyxin was not immunoprecipitated with WASP indicating the specificity of the approach. As negative controls, immunoprecipitates were prepared using an antibody to a bacterial protein (anti ActA 358C1 monoclonal antibody); these immunoprecipitates did not contain Fyb/SLAP, SLP-76 and Nck. To investigate whether the complex composed of Fyb/SLAP, SLP-76, Nck and WASP formed upon activation of T-cells, T-cell lysates from non-stimulated cells were immunoprecipitated with the WASP monoclonal and probed with antibodies to Fyb/SLAP, SLP-76, and Nck. Surprisingly, these proteins could be detected in these precipitates indicating that they form a complex also in non-stimulated T-cells. However, the relative amount of the complex in stimulated T-cells seemed to be higher than that in non-stimulated cells suggesting that the formation of such complex was, in part, stimulation-dependent. WASP immunoprecipitates of non-stimulated T-cells lysates probed with an antibody to phosphotyrosine showed three prominent bands running at 33, 76 and 95 kDa. These bands were also detected in WASP immunoprecipitates of stimulated T-cells lysates with, in addition, three bands running at 36, 47, and 70 kDa. Given that Nck and SLP-76 are phosphorylated in stimulated T-cells, the bands running at 47 and 76 kDa may correspond to these proteins. Taken together, these data show that Fyb/SLAP, SLP-76, Nck and WASP form a complex in T-cells and that the formation of this complex is enhanced upon stimulation of T-cells.

### Example 7

The effect of Fyb/SLAP protein on macrophage activation is examined as described above for t cells. Using these methods, it is determined that Fyb/SLAP is critical for macrophage activity.

### REFERENCES

Abel, K., Lingnau, A., Niebuhr, K., Wehland, J., and U. Walter.1996. Monoclonal antibodies against the focal adhesion protein VASP revealing epitopes involved in the interaction with two VASP binding proteins and VASP phosphorylation. Eur. J Cell Biol. 69, (Suppl. 42): 39a.

Arpin, M., Pringoult, E., Finidori, J., Garcia, A., Jeltsch, J. -M., Vandekerckhove, J., and D. Louvard. 1988. Sequence of human villin: A large duplicated domain homologous with other actin-severing proteins and a unique small carboxy-terminal domain related to villin specificity. J. Cell Biol. 107: 1759-1766.

Beckerle, M. C. 1997. Zyxin: zinc fingers at sites of cell adhesion. BioEssays 19:949-957.

Brindle, N. P. J., Holt, M. R., Davies, J. E., Price, C. J., and D. R. Critchley. 1996. The focal-adhesion vasodilator-stimulated phosphoprotein (VASP) binds to the proline-rich domain in vinculin. Biochem. J. 318:753-757.

Bubeck Wardenburg, J., Pappu, R., Bu, J. -Y., Mayer, B., Chemoff, J., Strauss, D., and A.C Chan. 1998. Regulation of PAK activation and the T-cell cytoskeleton by the linker protein SLP-76. Immunity 9:607-616.

Carl, U. D., Pollmann, M., Orr, E., Gertler, F. B., Chakraborty, T., and J. Wehland. 1999. Aromatic and basic residues within the EVH1 domain of VASP specify its interaction with proline-rich ligands. Curr. Biol. 9:715-718.

Chakraborty, T., Ebel, F., Domann, E., Niebuhr, K., Gerstel, B., Pistor, S., Temm-Grove, C. J., Jockusch, B. M., Reinhard, M., Walter, U., and J. Wehland. 1995. A focal adhesion factor directly linking intracellularly motile Listeria monocytogenes and Listeria ivanovii to the actin-based cytoskeleton of mammalian cells. EMBO J. 14:1314-1321.

Elements, J. L., Boerth, N. J., Lee, J. R., and Koretzky, G. A. 1999. Integration of T cell receptor dependent signaling pathways by adapter proteins Annu. Rev. Immunol. 17:89-108.

Domann, E., Wehland, J., Rohde, M., Pistor, S., Hartl, M., Goebel, W., Leimeister-Wächter, M., Wuenscher, M., and T. Chakraborty. 1992. A novel bacterial virulence gene in Listeria monocytogenes required for host cell microfilament interaction with homology to the proline-rich region of vinculin. EMBO J. 11:1981-1990.

Frank, R. 1992. Spot synthesis: An easy technique for the positionally addressable, parallel chemical synthesis on a membrane support. Tetrahedron. 48:9217-9232.

Geiger, B., Rosen, D., and G. Berke. 1982. Spatial relationships of microtubule-organizing centers and the contact area of cytotoxic T-lymphocytes and target cells. J.Cell Biol. 95:137-143.

Gertler, F. B., Niebuhr, K., Reinhard, M., Wehland, J., and P. Soriano. 1996. Mena, a relative of VASP and Drosophila Enabled, is implicated in the control of microfilament dynamics. Cell. 87:227-239.

Jockusch, B. M., Bubeck, P., Giehl, K., Kroemker, M., Moschner, J., Rothkegel, M., Rüdiger, M., Schlüter, K., Stanke, G., and J. Winkler. 1995. The molecular architecture of focal adhesions. Annu. Rev. Cell Dev. Biol. 11:379-416.

Kreft, J., Dumbsky, M., and S. Theiss. 1995. The actin-polymerization protein from Listeria ivanovii is a large repeat protein which shows only limited amino acid sequence homology to ActA from Listeria monocytogenes. FEMS Microbiol. Lett. 126:113-122.

Kupfer, A., Swain, S. L., and S. J. Singer. 1987. The specific interaction of helper T cells and antigen-presenting B cells. J Exp. Med. 165:1565-1580.

Kupfer, A., Mosmann, T. R., and H. Kupfer. 1991. Polarized expression of cytokines in cell conjugates of helper T-cells and splenic B cells. Proc. Natl. Acad. Sci. USA 88:775-779.

Lanier, L. M., Gates, M. A., Witke, W., Menzies, S., Wehman, A. M., Macklis, J. D., Kwiatkowski, D., Soriano, P., and F. B. Gertler. 1999. Mena is required for neurulation and commissure formation. Neuron. 22:313-325.

Lasa, I., David, V., Gouin, E., Marchand, J. -B., and P. Cossart. 1995. The amino-terminal part of ActA is critical for the actin-based motility of Listeria monocytogenes; the central proline-rich region acts as a stimulator. Mol. Microbiol. 18:425-436.

Lasa, I., Gouin, E., Goethals, M., Vancompernolle, K., David, V., Vandekerckhove, J., and P Cossart. 1997. Identification of two regions in the N-terminal domain of ActA involved in the actin comet tail formation by Listeria monocytogenes. EMBO J. 16:1531-1540.

Lowin-Kropf, B., Smith Shapiro, V., and A. Weiss. 1998. Cytoskeletal polarization of T-cells is regulated by an immunoreceptor tyrosine-based activation motif-dependent mechanism. J. Cell Biol. 140:861-871.

Machesky, L. M., and R. H. Insall. 1998. Scar1 and the related Wiskott-Aldrich syndrome protein, WASP, regulate the actin cytoskeleton through the Arp2/3 complex. Curr. Biol. 8:1347-1356.

May, R. C., Hall, M. E., Higgs, H. N., Pollard, T. D., Chakraborty, T., Wehland, J., Machesky, L. M., and A. S. Sechi. 1999. The Arp2/3 complex is essential for the actin-based motility of Listeria monocytogenes. Curr. Biol. 9:759-762.

Mies, B., Rottner, K., and Small J. V. Multiple immunofluorescence microscopy of the cytoskeleton. In Cell Biology: a laboratory handbook, Celis, J. E., Ed. Academic Press, San Diego, Calif., 1998, pp. 469-476.

Musci, M. A., Hendricks-Taylor, L. R., Motto, D. G., Paskind, M., Kamens, J., Turck, Ch. W., and G. A. Koretzky. 1997. Molecular cloning of SLAP-130, an SLP-76-associated substrate of the T cell antigen receptor-stimulated protein tyrosine kinases. J. Biol. Chem. 272:11674-11677.

Niebuhr, K., Chakraborty, T., Rhode, M., Gazlig, T., Jansen, B., Kollner, P., and J. Wehland. 1993. Localization of the ActA polypeptide of Listeria monocytogenes in infected tissue culture cell lines: ActA is not associated with actin comets. Infect. Immun. 61:2693-2802.

Niebuhr, K., Ebel, F., Frank, R., Reinhard, M., Domann, E., Carl, U. D., Walter, U., Gertler, F. B., Wehland, J., and T. Chakraborty. 1997. A novel proline-rich motif present in ActA of Listeria monocytogenes and cytoskeletal proteins is the ligand for the EVH1 domain, a protein module present in the Ena/VASP family. EMBO J. 16:5433-5444.

Niebuhr, K. Lingnau, A., Frank, R., and J. Wehland. 1998. Rapid procedures for preparing monoclonal antibodies and identifying their epitopes. In Cell Biology: A Laboratory Handbook (J. Celis Ed.), Academic Press, San Diego, Vol. 2, 398-403.

Penninger, J. M., and G. R. Crabtree. 1999. The actin cytoskeleton and lymphocyte activation. Cell. 96:9-12.

Pistor, S., Chakraborty, T., Walter, U., and J. Wehland. 1995. The bacterial actin nucleator protein ActA of Listeria monocytogenes contains multiple binding sites for host microfilament proteins. Curr. Biol. 5:517-525.

Reinhard, M., Halbrügge, M., Scheer, U., Wiegand, Ch., Jockusch, B. M., and U. Walter. 1992. The 46/50 kDa phosphoprotein VASP purified from human platelets is a novel protein associated with actin filaments and focal contacts. EMBO J. 11:2063-2070.

Reinhard, M., Rüdiger, M., Jockusch, B. M., and U. Walter. 1996. VASP interaction with vinculin: a recurring theme of interactions with proline-rich motifs. FEBS Lett. 399:103-7.

Rivero-Lezcano, O. M., Marcilla, A., Sameshima, J. H., and K. C. Robbins. 1995. Wiskott-Aldrich Syndrome Protein physically associates with Nck through Src homology 3 domains. Mol. Cell. Biol. 15:5725-5731.

Ryser. J. -E., Rungger-Brändle, E., Chaponnier, C., Gabbiani, G., and P. Vassalli. 1982. The area of attachment of cytotoxic T lymphocytes to their target cells shows high motility and polarization of actin, but not myosin. J Immun. 128:1159-1162.

Sattler, M., Durstin, M. A., Frank, D. A., Okuda, K., Kaushansky, K., Salgia, R., and J. D. Griffin. 1995. The thrombopoietin receptor c-MPL activates JAK2 and TYK2 tyrosine kinases. Exp. HematoL 23:1040-1048.

Symons, M., Derry, J. M. J., Karlak, B., Jiang, S., Lemahieu, V., McCormick, F., Francke, U., and A. Abo. 1996. Wiskott-Aldrich Syndrome Protein, a novel effector for the GTPase CDC42Hs, is implicated in actin polymerization. Cell. 84:723-734.

Takubo, T., Hino, M., Suzuki, K., and N. Tatsumi. 1998. Localization of myosin, actin, alpha-actinin, tropomyosin and vinculin in surface-activated, spreading human platelets. Biotech. Histochem. 73:310-315.

Valitutti, S., Dessing, M., Aktories, K., Gallati, H., and A. Lanzavecehia. 1995. Sustained signaling leading to T-cell activation results from prolonged T-cell receptor occupancy. Role of T-cell actin cytoskeleton. J.Exp.Med. 181:577-584.

Van Troys, M., Dewitte, D., Goethals, M., Carlier, M. -F., Vanderkerckhove, J., and C. Ampe. 1996. The actin binding site of thymosin (4 mapped by mutational analysis. EMBO J. 15:201-210.

Veale, M., Raab, M., Li, Z., da Silva, A. J., Kraeft, S. K., Weremowicz, S., Morton, C. C., and C. E. Rudd 1999. Novel isoform of lymphoid adaptor FYN-T-binding protein (FYB-130) interacts with SLP-76 and Up-regulates interleukin 2 production. J Biol Chem 274:28427-28435.

Equivalents

All references disclosed herein are incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for inhibiting cytoskeletal rearrangement in a cell or cell fragment, comprising:
contacting the cell or cell fragment with an amount of a Fyb/SLAP complex inhibitor sufficient to inhibit the formation of a complex of an Ena/VASP protein and a Fyb/SLAP protein.

2. The method of claim 1, wherein the Fyb/SLAP complex inhibitor binds to the EVH1 domain of the the Ena/VASP protein and inhibits binding of the Ena/VASP protein to a Fyb/SLAP protein.

3. The method of claim 2, wherein the Ena/VASP family protein is selected from the group consisting of Ena, Mena, VASP and Ev1.

4. The method of claim 2, wherein the Fyb/SLAP complex inhibitor comprises the peptide FPPPP (SEQ ID NO: 15) or a peptide mimetic having an equivalent binding specificity.

5. The method of claim 4, wherein the Fyb/SLAP complex inhibitor is selected from the group consisting of ActA repeats, EVH1 binding peptides, ScarWA, and dominant negative Fyb/SLAP fragments.

6. The method of claim 1, wherein the Fyb/SLAP complex inhibitor binds a Fyb/SLAP protein and inhibits binding of an Ena/VASP protein to the Fyb/SLAP protein.

7. The method of claim 6, wherein the Fyb/SLAP complex inhibitor is a polypeptide which has a functional EVH1 domain but which cannot function as an Ena/VASP protein in cytoskeletal rearrangement of the cell or cell fragment.

8. The method of claim 6, wherein the Fyb/SLAP complex inhibitor is an antibody or antibody fragment which binds Fyb/SLAP.

9. The method of claim 1, wherein the Fyb/SLAP complex inhibitor is an antisense nucleic acid molecule which inhibits the expression of a Fyb/SLAP protein or an Ena/VASP protein.

10. The method of claim 1, wherein the Fyb/SLAP complex inhibitor is administered in vivo to a subject in need of such treatment.

11. The method of claim 1, wherein the cell is a lymphocyte or a macrophage.

12. The method of claim 11, wherein the lymphocyte is a T cell.

13. The method of claim 1, wherein the cell fragment is a platelet.

14. A method for inhibiting cytoskeletal rearrangement in a cell or cell fragment, comprising:
inhibiting the expression of a Fyb/SLAP protein sufficiently to inhibit the formation of a complex of an Ena/VASP protein and a Fyb/SLAP protein.

15. A method for enhancing cytoskeletal rearrangement in a cell or cell fragment, comprising:
contacting the cell or cell fragment with an amount of a composition which increases the amount of a Fyb/SLAP polypeptide in the cell or cell fragment sufficient to enhance the formation of a complex of an Ena/VASP protein and a Fyb/SLAP protein

16. The method of claim 15, wherein the composition is a Fyb/SLAP polypeptide.

17. The method of claim 15, wherein the composition is a nucleic acid molecule which encodes a Fyb/SLAP polypeptide.

18. The method of claim 15, wherein the composition which increases the amount of a Fyb/SLAP polypeptide is administered to a subject in need of such treatment in vivo.

19. The method of claim 15, wherein the cell is a lymphocyte or a macrophage.

20. The method of claim 19, wherein the lymphocyte is a T cell.

21. The method of claim 15, wherein the cell fragment is a platelet.

22. A method for increasing a T cell response to T cell receptor stimulation, comprising:
contacting a T cell with a Fyb/SLAP complex activator sufficient to promote the formation of a complex of an Ena/VASP protein and a Fyb/SLAP protein in the T cell.

23. The method of claim 22, wherein the Fyb/SLAP activator is administered in vivo to a subject in need of such treatment.

24. The method of claim 23, wherein the subject has or is at risk of developing an infectious disease.

25. The method of claim 23, wherein the subject has or is at risk of developing cancer.

26. A method for inhibiting a T cell response to T cell receptor stimulation, comprising:
contacting a T cell with an amount of a Fyb/SLAP complex inhibitor sufficient to inhibit formation of a complex of a Fyb/SLAP protein and an Ena/VASP protein in the T cell.

27. The method of claim 26, wherein the Fyb/SLAP inhibitor is administered in vivo to a subject in need of such treatment.

28. The method of claim 27, wherein the subject has or is at risk of developing an autoimmune disease.

29. The method of claim 27, wherein the subject has or is at risk of developing a condition characterized by inflammation.

30. The method of claim 26, wherein the Fyb/SLAP complex inhibitor binds to the EVH1 domain of the the Ena/VASP protein and inhibits binding of the Ena/VASP protein to a Fyb/SLAP protein.

31. The method of claim 30, wherein the Ena/VASP family protein is selected from the group consisting of Ena, Mena, VASP and Ev1.

32. The method of claim 30, wherein the Fyb/SLAP complex inhibitor comprises the peptide FPPPP (SEQ ID NO: 15) or a peptide mimetic having an equivalent binding specificity.

33. The method of claim 32, wherein the Fyb/SLAP complex inhibitor is selected from the group consisting of ActA repeats, EVH1 binding peptides, ScarWA, and dominant negative Fyb/SLAP fragments.

34. The method of claim 26, wherein the Fyb/SLAP complex inhibitor binds a Fyb/SLAP protein and inhibits binding of an Ena/VASP protein to the Fyb/SLAP protein.

35. The method of claim 34, wherein the Fyb/SLAP complex inhibitor is a polypeptide which has a functional EVH1 domain but which cannot function as an Ena/VASP protein in cytoskeletal rearrangement of the cell or cell fragment.

36. The method of claim 34, wherein the Fyb/SLAP complex inhibitor is an antibody or antibody fragment which binds Fyb/SLAP.

37. The method of claim 26, wherein the Fyb/SLAP complex inhibitor is an antisense nucleic acid molecule which inhibits the expression of a Fyb/SLAP protein or an Ena/VASP protein.

38. The method of claim 26, wherein the Fyb/SLAP complex inhibitor is administered in vivo to a subject in need of such treatment.

39. A method for increasing platelet aggregation, comprising:
contacting a platelet with Fyb/SLAP complex inhibitor to inhibit formation of a complex of a Fyb/SLAP protein and an Ena/VASP protein in the platelet.

40. The method of claim 39, wherein the Fyb/SLAP inhibitor is administered in vivo to a subject in need of such treatment.

41. The method of claim 40, wherein the administration of the Fyb/SLAP inhibitor increases wound healing or clotting.

42. The method of claim 39, wherein the Fyb/SLAP complex inhibitor binds to the EVH1 domain of the the Ena/VASP protein and inhibits binding of the Ena/VASP protein to a Fyb/SLAP protein.

43. The method of claim 42, wherein the Ena/VASP family protein is selected from the group consisting of Ena, Mena, VASP and Ev1.

44. The method of claim 42, wherein the Fyb/SLAP complex inhibitor comprises the peptide FPPPP (SEQ ID NO:15) or a peptide mimetic having an equivalent binding specificity.

45. The method of claim 44, wherein the Fyb/SLAP complex inhibitor is selected from the group consisting of ActA repeats, EVH1 binding peptides, ScarWA, and dominant negative Fyb/SLAP fragments.

46. The method of claim 39, wherein the Fyb/SLAP complex inhibitor binds a Fyb/SLAP protein and inhibits binding of an Ena/VASP protein to the Fyb/SLAP protein.

47. The method of claim 46, wherein the Fyb/SLAP complex inhibitor is a polypeptide which has a functional EVH1 domain but which cannot function as an Ena/VASP protein in cytoskeletal rearrangement of the cell or cell fragment.

48. The method of claim 46, wherein the Fyb/SLAP complex inhibitor is an antibody or antibody fragment which binds Fyb/SLAP.

49. The method of claim 39, wherein the Fyb/SLAP complex inhibitor is an antisense nucleic acid molecule which inhibits the expression of a Fyb/SLAP protein or an Ena/VASP protein.

50. The method of claim 39, wherein the Fyb/SLAP complex inhibitor is administered in vivo to a subject in need of such treatment.

51. A composition comprising an effective amount of a Fyb/SLAP complex inhibitor and a pharmaceutically acceptable carrier.

52. The composition of claim 51, wherein the Fyb/SLAP complex inhibitor comprises the peptide FPPPP (SEQ ID NO: 15) or a peptide mimetic having an equivalent binding specificity.

53. The composition of claim 52, wherein the Fyb/SLAP complex inhibitor is selected from the group consisting of ActA repeats, EVH1 binding peptides, ScarWA, and dominant negative Fyb/SLAP fragments.

54. The composition of claim 51, wherein the Fyb/SLAP complex inhibitor binds a Fyb/SLAP protein and inhibits binding of an Ena/VASP protein to the Fyb/SLAP protein.

55. The composition of claim 54, wherein the Fyb/SLAP complex inhibitor is a polypeptide which has a functional EVH1 domain but which cannot function as an Ena/VASP protein in cytoskeletal rearrangement of the cell or cell fragment.

56. The composition of claim 54, wherein the Fyb/SLAP complex inhibitor is an antibody or antibody fragment which binds Fyb/SLAP.

57. The composition of claim 51, wherein the Fyb/SLAP complex inhibitor is an antisense nucleic acid molecule which inhibits the expression of a Fyb/SLAP protein or an Ena/VASP protein.

58. A composition comprising an effective amount of a Fyb/SLAP complex activator and a pharmaceutically acceptable carrier.

59. The composition of claim 58, wherein the Fyb/SLAP complex activator is a Fyb/SLAP polypeptide or a nucleic acid molecule which encodes a Fyb/SLAP polypeptide.

60. An isolated antibody which selectively binds human Fyb/SLAP 2 but not Fyb/SLAP 1.

61. The isolated antibody of claim 60, wherein the antibody binds to SEQ ID NO:7.

62. The isolated antibody of claim 60, wherein the antibody binds to an epitope defined by amino acids 637-682 of SEQ ID NO:4.

63. An isolated antibody which selectively binds Arp3 polypeptide, wherein the antibody binds to an epitope formed by the amino acids set forth in SEQ ID NO: 11.

64. An isolated human Fyb/SLAP2 polypeptide comprising the amino acids 637-682 of SEQ ID NO:4.

65. The isolated human Fyb/SLAP2 polypeptide of claim 64, wherein the amino acid sequence comprises SEQ ID NO:4

66. An isolated nucleic acid molecule which encodes the human Fyb/SLAP2 polypeptide of claim 64.

67. The isolated nucleic acid molecule of claim 66, wherein the nucleic acid molecule comprises nucleotides 1939-2076 of of SEQ ID NO:5.

68. The isolated nucleic acid molecule of claim 66, wherine the nucleic acid molecule comprises the nucleotide sequence of SEQ ID NO:5.

69. An expression vector comprising the isolated nucleic acid molecule of any of claims 66-68 operably linked to a promoter.

70. A host cell transformed or transfected with the expression vector of claim 69.

71. A method for identifying lead compounds for a pharmacological agent useful in the treatment of disease associated with Fyb/SLAP-Ena/VASP complex formation, comprising
forming a mixture comprising a Fyb/SLAP protein or EVH1 domain binding fragment thereof, a Ena/VASP protein or Fyb/SLAP binding fragment thereof, and a candidate pharmacological agent,
incubating the mixture under conditions which, in the absence of the candidate pharmacological agent, permit a first amount of specific binding of the Fyb/SLAP protein or EVH1 domain binding fragment thereof and the Ena/VASP protein or Fyb/SLAP binding fragment thereof, and
detecting a test amount of the specific binding of the Fyb/SLAP protein or EVH1 domain binding fragment thereof and the Ena/VASP protein or Fyb/SLAP binding fragment thereof, wherein an increase in the test amount of specific binding in the presence of the candidate pharmacological agent relative to the first amount of specific binding indicates that the candidate pharmacological agent is a lead compound for a pharmacological agent which increases specific binding of the Fyb/SLAP protein or EVH1 domain binding fragment thereof and the Ena/VASP protein or Fyb/SLAP binding fragment thereof, and wherein a decrease in the test amount of specific binding in the presence of the candidate pharmacological agent relative to the first amount of specific binding indicates that the candidate pharmacological agent is a lead compound for a pharmacological agent which decreases specific binding of the Fyb/SLAP protein or EVH1 domain binding fragment thereof and the Ena/VASP protein or Fyb/SLAP binding fragment thereof.

72. A method for identifying lead compounds for a pharmacological agent useful in the treatment of disease associated with Fyb/SLAP-Ena/VASP complex formation, comprising
contacting a Fyb/SLAP protein or EVH1 domain binding fragment thereof with a candidate pharmacological agent, and
determining the binding of the candidate pharmacological agent to the Fyb/SLAP protein or EVH1 domain binding fragment thereof.
